(19) United States
(12) Patent Application Publication
(10) Pub. No.: US 2016/0067444 A1
(43) Pub. Date: Mar. 10, 2016

(54) A Catheter Having an Expansile Sheath

(71) Applicant: NATIONAL UNIVERSITY OF IRELAND, GALWAY, Galway (IE)

(72) Inventors: Wayne ALLEN, Furbo (IE); Mark BRUZZI, Galway (IE); Colin FORDE, Kinvara (IE); Liam MULLINS, Athlone (IE)

(73) Assignee: NATIONAL UNIVERSITY OF IRELAND, GALWAY, Galway (IE)

(21) Appl. No.: 14/775,045

(22) PCT Filed: Mar. 12, 2014
(86) PCT No.: PCT/EP2014/054829

§ 371 Date: Sep. 11, 2015

Related U.S. Application Data
(60) Provisional application No. 61/787,268, filed on Mar. 15, 2013.

(51) Int. Cl. A61M 25/00

(52) U.S. Cl. CPC A61M 25/0023; A61M 25/005; A61M 25/0045; A61M 25/0074; A61M 2025/0024; A61M 2025/0025; A61M 2025/0035; A61M 2025/0098; A61M 2025/09008

(57) ABSTRACT

A catheter includes a shaft extending from a proximal end to a distal end and a sheath which extends along the length of the catheter shaft. The sheath includes a compliant material and is expansile for fluid delivery from a distal end of the sheath to the proximal end of the catheter. The sheath is connected to a Luer on the proximal end of the catheter. Upon injection of fluid/particles through the Luer, the sheath expands to provide a temporary lumen which facilitates flow of fluid/particles from the proximal end of the catheter to an opening or opening adjacent to the distal end of the sheath and at or near the distal end of the catheter shaft for delivery of the fluid/particles into the vessel. Upon cessation of injection pressure, the sheath automatically contracts to provide a lower profile to easily accommodate repositioning/removal of the catheter.

This invention relates to catheters.

Catheters with a balloon at the distal end thereof through which a contrast fluid is delivered are known. In some cases the balloon has a valve located at the proximal end of the balloon. The valve is opened by means of a sliding mechanism connecting between the valve and a handle external of the patient at a proximal end of the catheter. An operator controls fluid delivery through manipulation of the proximal handle mechanism. Fluid is delivered through a lumen in the catheter which lumen is also used for balloon inflation.

In current balloon catheter procedures (including stenting) a guide catheter is placed proximally to the target site. This acts as a conduit for delivery of the balloon catheter and contrast media. A guide wire is delivered through the guide catheter and the balloon catheter is delivered over the guide wire to the target site and the balloon is inflated. To deliver contrast medium the guide wire must be removed from the balloon catheter, or, the balloon catheter may be removed and contrast delivered through the guide catheter.

In some current diagnostic procedures (angiogram)/drug/therapeutic/biologic/embolic material delivery the catheter requires the support of a guide wire to reach the target site, especially if fluid delivery is required at multiple sites. In these instances the guide wire must be removed to allow delivery of the contrast fluid. After the contrast fluid is delivered the catheter is generally flushed with saline and the guide wire re-inserted.

One problem with existing technology is that excessive procedural time is required because removal of the guide wire from the balloon catheter is required for each injection of contrast fluid. Removal of the guide wire from the guide catheter/introducer catheter is also required for each injection. Removal of the catheter (such as a balloon catheter) from the guide catheter/introducer sheath may also be required for each injection.

Another problem with known catheter technology is the loss of original device position because removal of the guide wire from the balloon catheter, removal of the guide wire from the guide catheter/introducer catheter and removal of the catheter (e.g. balloon catheter) from the guide catheter/introducer sheath are required for each injection.

Further with each insertion and/or removal of a device, such as a guide wire, guide catheter and the like, there is a risk of damage to the intima of the vessel due to contact with the device.

In some cases the balloon is deflated during contrast fluid delivery as fluid delivery and balloon inflation are controlled through the same lumen. The balloon loses pressure (can be inflation or vacuum pressure) when the valve is opened to deliver fluid.

A further problem is that higher than necessary volumes of contrast medium is delivered; With balloon catheters, the contrast fluid is delivered at the distal end of the guide catheter/introducer sheath or the distal end of the balloon catheters (after the balloon catheter is withdrawn proximally of the target site) and both the guide catheter/introducer sheath and the balloon catheter distal ends may not be positioned directly proximal to the target site, resulting in a higher volume contrast injection to ensure adequate contrast reaches the target site.

With guide catheters, excessive volumes of contrast are also delivered each time the catheter lumen requires flushing with saline prior to reinsertion of the guide wire. In this case the residual volume of contrast contained within the lumen of the catheter is flushed into the vessel. If the guide catheter tip is dislodged during removal/insertion of the guide wire, extra contrast may be required to guide the repositioning of the guide catheter.

Increased volume of contrast can lead to nephrotoxicity. Certain patient cohorts are particularly susceptible to this adverse effect e.g. End-stage renal disease patient.

There may also be relatively high radiation exposure as more fluoroscopic images and longer imaging time are required to find the original location. The addition of a new lumen of a catheter has implications for profile and flexibility, making it undesirable for most procedures.

US-A-2011/282198 describes an angiographic catheter having an elongated flexible insertion tube and a cylindrical inflation sheath of polyamide (Nylon) mounted thereon. The two opposed ends of the sheath are heat sealed to the outside of the insertion tube. An extension tube is passed into the proximal end and is snap-fitted to a syringe. The syringe contains radiographic fluid and is arranged to deliver the fluid through one or more discharge ports at the distal end. When the procedure is completed the sheath is deflated using the syringe.

There are a number of problems with the system described in US2011/282198A. An adjustment to the Nylon sheath is required initially to achieve a compact package (low profile) for insertion into the body. The sheath is then inflated to enable fluid flow through the sheath. When the procedure is complete further adjustment (deflation) is required in achieve a compact package.

Compared to many other polymers Nylon is stiff. This means that in order to increase in diameter, a large pressure (force) will be required. The material cannot undergo excessive increases in diameter without a very high pressure, or potential for bursting. The sheath described in US2011/282198A must first be adjusted or folded down into a collapsed configuration for insertion. Once the adjustment is made in the polyamide sheath, the material will take a crinkled configuration which increases friction during insertion, removal and position adjustment.

### STATEMENTS OF INVENTION

According to the invention there is provided a catheter comprising a shaft extending from a proximal end to a distal end and a sheath which extends substantially along the full length of the catheter shaft, the sheath having a proximal end and a distal end and being expansile for fluid delivery from the proximal end of the sheath to the distal end of the sheath, the sheath having a contracted configuration in which the sheath lies substantially against the outer wall of the catheter shaft and an expanded delivery configuration in which at least a portion of the sheath extends radially outwardly of the catheter shaft, wherein the sheath comprises a compliant material to urge the sheath to return automatically from the expanded configuration to the contracted configuration.

In one embodiment the sheath comprises a plurality of layers. The sheath may comprise an outer layer of compliant material and an inner layer of a reinforcing material. The reinforcing material is at least partially porous.

In one case there is an inner sealing layer between the reinforcing layer and the outer wall of the catheter.

In one case the reinforcing layer lies between the inner sealing layer and the outer elastic layer.

In one embodiment wherein the reinforcing layer of the sheath comprises a non-compliant material.

The distal end of the sheath is preferably secured to the catheter shaft. The distal end of the sheath is bonded to the catheter shaft.

In one embodiment the proximal end of the sheath is connected to a Luer fitting to provide access for fluid flow into the sheath. The sheath may be spaced-apart from the shaft at the proximal end to define an in-flow region for fluid. There may be a protector between the Luer fitting and the proximal end of the sheath. The protector may comprise a protective sleeve or a support tube.

In one case the protector extends distally of the Luer fitting at the proximal end and is adapted to provide strain relief at the connection between the Luer fitting and the sheath.

In one embodiment the catheter shaft and the sheath are concentric in the expanded delivery configuration.

In one case the catheter shaft and the sheath are concentric in the contracted configuration and eccentric in the expanded configuration.

A catheter as claimed in any of claims 1 to 16 wherein the sheath is eccentric to the shaft in both the contracted and the expanded configuration.

In one embodiment the catheter is generally cylindrical having an outer shaft diameter and the sheath is generally cylindrical having an inner sheath diameter and wherein the outer shaft diameter is substantially equal to the inner sheath diameter.

The sheath may comprise at least one fluid outlet or outlets adjacent to the distal end of the sheath. The sheath may comprise a plurality of outlets or the sheath comprises a single outlet on one side of the sheath. The sheath in the region of a single outlet may be adapted to be locally inflatable for delivery of fluid into a side vessel.

In one embodiment comprises a deflector element for directing fluid flow from the outlet(s). The deflector element may be adapted to deflect fluid to flow in a proximal direction.

In one case the catheter comprises at least one radiopaque marker. The sheath may include radiopaque markers to define rotational orientation of the catheter.

In one embodiment the catheter comprises means to prevent adhesion between the outer surface of the catheter shaft and the inner surface of the sheath. There may be a coating on the outer surface of the catheter shaft and/or the inner surface of the sheath. The coating may be of a lubricious material.

In one case the adhesion prevention means comprises formations on the outer surface of the shaft and/or the inner surface of the sheath.

The catheter shaft may comprise a lumen. The catheter shaft may not comprise a lumen.

In one case the catheter shaft comprises a guide wire.

Also provided is a catheter comprising a shaft extending from a proximal end to a distal end and a sheath which extends substantially along the full length of the catheter shaft, the sheath having a proximal end and a distal end and being expansile for fluid delivery from the proximal end of the sheath to the distal end of the sheath.

In one embodiment the sheath has a contracted configuration in which the sheath lies substantially against the outer wall of the catheter shaft and an expanded delivery configuration in which at least a portion of the sheath extends radially outwardly of the catheter shaft.

In one case the catheter shaft and the sheath are concentric. In another case the catheter shaft and sheath are concentric when in the contracted configuration and eccentric when in the expansile configuration.

The catheter may be generally cylindrical having an outer shaft diameter and the sheath is generally cylindrical having an inner sheath diameter and wherein the outer shaft diameter is substantially equal to the inner sheath diameter.

In one embodiment the sheath comprises a fluid outlet, or outlets, adjacent to the distal end of the sheath. In one embodiment this outlet consists of multiple holes along the distal end of the sheath. In one embodiment the outlet is a single opening on one side of the sheath. In one embodiment the outlet is locally inflatable to inflate into side vessel for accurate delivery into a side vessel. In one embodiment the outlet(s) are covered by an expandable collar originating at the distal tip. This collar expands with the sheath and prevents flow of fluid distally to redirect flow proximally.

The sheath may comprise a plurality of layers. These may be of different materials. The sheath may comprise an outer layer of elastic material and an inner layer of a reinforcing material (or vice versa). The reinforcing material may be at least partially porous. There may be an inner sealing layer between the reinforcing layer and the outer wall of the catheter. The reinforcing layer may lie between the inner sealing layer and the outer elastic layer. In one embodiment each layer, when expanded, enables a separate fluid delivery lumen. In one embodiment at least one of the layers is radio opaque. In one embodiment radio opaque markers are incorporated to define rotational orientation of the catheter. In one embodiment the outer layer of the sheath is of a compliant material which may be an elastomer such as silicone, urethane or Pebax. In one embodiment the wall thickness of the sheath would be <0.005″. In one embodiment the reinforcing layer of the sheath comprises a non-compliant material. In one embodiment the compliant layer consists of sections of different expansile properties. In one aspect the distal end of the sheath is secured to the catheter shaft. In one aspect the distal end is not secured to the catheter shaft. The distal end of the sheath may be bonded to the catheter shaft. Alternatively or additionally a marker band is provided at the distal end of the sheath. In one embodiment the sheath may be attached to the catheter at various points along the length. In one embodiment the sheath may be attached to the catheter in a single line along the length creating a spine. In one embodiment the catheter comprises means to prevent adhesion between the outer surface of the catheter shaft and the inner surface of the sheath. There may be a coating on the outer surface of the catheter shaft and/or the inner surface of the sheath. The coating may be of a lubricious material. In one embodiment the sheath comprises a low friction coating on the outer surface. The coating may be of a lubricious material. In one embodiment the sheath comprises a low friction material embedded within the sheath. The coating may be of a lubricious material. In another embodiment the adhesion prevention means comprises formations on the outer surface of the shaft and/or the inner surface of the sheath. In one embodiment the adhesion prevention means comprises charging the surfaces surface charge on the sheath and catheter.

In one embodiment the catheter outer surface or the sheath inner surface comprise helical pattern to aid the mixing of two fluids.

### Manufacturing

The sheath may be stretched longitudinally during assembly to prevent wrinkling and/or to tune expansile behaviour.

The sheath may be added to the catheter by using one or a combination of the following mechanisms:
threading the catheter through the sheath,
coextrusion of the sheath onto the catheter, inflation of the sheath to facilitate sliding the sheath onto the catheter,
applying vacuum to the outside of the sheath to increase its diameter and facilitate sliding onto the catheter,
applying vacuum to the inside of the catheter to reduce its diameter and facilitate sliding onto the catheter,
roll the sheath onto the catheter, stretch the catheter to reduce its diameter and facilitate sliding the sheath onto the catheter, dip coat the catheter where the coating becomes the sheath,
over mold the sheath onto the catheter, spray coat the sheath onto the catheter; or
vapour deposition of the sheath onto the catheter.

### BRIEF DESCRIPTION OF THE DRAWINGS

The invention will be more clearly understood from the following description of an embodiment thereof, given by way of example only, with reference to the accompanying drawings, in which:

FIG. 1 is a perspective view of a catheter according to the invention in one configuration of use;

FIG. 2 is a perspective view of the catheter of FIG. 1 in another configuration of use;

FIG. 3 is an enlarged view of a distal end of the catheter of FIG. 2;

FIG. 4 is another perspective of the catheter in a balloon expanded configuration;

FIG. 5 is an enlarged view of the distal end of the catheter in a balloon retracted configuration;

FIG. 6 is a perspective view of the catheter in a sheath expanded configuration;

FIG. 7 is an enlarged view of the catheter of FIG. 6;

FIG. 8 is another perspective view of a distal end of the catheter in a balloon expanded and sheath relaxed configuration;

FIG. 9 is a perspective view of the distal end of the catheter in a balloon retracted and sheath expanded configuration;

FIG. 10 is a longitudinal across sectional view of a percutaneous transluminal angioplasty (PTCA) catheter with an external sheath relaxed;

FIG. 11 is a cross sectional view of the PTCA catheter of FIG. 10 with the external sheath expanded;

FIGS. 12 and 13 are transverse cross sectional view of a catheter of the invention with an outer sheath in relaxed and expanded configurations respectively;

FIGS. 14 and 15 are longitudinal cross sectional views of a distal end of a catheter of the invention with an outer sheath in relaxed and expanded configurations respectively;

FIGS. 16 and 17 are longitudinal cross sectional views of a distal end of a catheter of the invention with an outer sheath in relaxed and expanded configurations respectively;

FIGS. 18 to 21 are longitudinal cross sectional views of a proximal end of catheters according to the invention;

FIG. 22 is a longitudinal cross sectional view of another catheter of the invention with the sheath in a contracted delivery configuration;

FIG. 23 is a cross sectional view on the line A-A in FIG. 22;

FIG. 24 is a longitudinal cross sectional view of the catheter of FIG. 22 with a sheath in the expanded configuration;

FIG. 25 is a cross sectional view on the line B-B in FIG. 24;

FIGS. 26 and 28 are transverse cross sectional views of another catheter of the invention with an outer sheath in relaxed and expanded configurations respectively;

FIG. 27 is an enlarged view of a detail of FIG. 26;

FIGS. 29 and 31 are a transverse cross sectional views of another catheter of the invention with an outer sheath in relaxed and expanded configurations respectively;

FIGS. 30 and 32 are enlarged views of details of FIGS. 29 and 31;

FIG. 33 is a perspective view of a catheter according to the invention which in this case is a guide catheter with a sheath relaxed and contracted;

FIG. 34 is an enlarged view of a detail of FIG. 33;

FIG. 35 is a perspective view of the catheter of FIG. 33 with the sheath expanded;

FIG. 36 is an enlarged view of a detail of FIG. 35;

FIG. 37 is a cross sectional view of a guide catheter according to the invention with an external sheath relaxed;

FIG. 38 is an enlarged view of a distal detail A of FIG. 37;

FIG. 39 is a longitudinal cross sectional view of the guide catheter of FIG. 37 with the external sheath expanded;

FIG. 40 is an enlarged view of a distal end detail B of FIG. 39;

FIGS. 41 and 42 are perspective views of another catheter according to the invention with a sheath in an expanded and a partially expanded configuration respectively;

FIG. 43 is a longitudinal cross sectional view of a catheter system with a sheath in a relaxed delivery configuration;

FIG. 44 is a cross sectional view on the line C-C in FIG. 43;

FIG. 45 is a longitudinal cross sectional view of the catheter system of FIG. 42 with a sheath in an expanded configuration;

FIG. 46 is a cross sectional view on the line D-D in FIG. 45;

FIG. 47 is a plan view of a catheter shaft showing indentations along the shaft;

FIG. 48 is a transverse cross sectional view of a catheter shaft showing indentations along the shaft;

FIGS. 49 and 50 are transverse cross sectional views of the catheter shaft of FIGS. 47 and 48 with an outer sheath in relaxed and expanded configurations respectively;

FIGS. 51 and 52 are longitudinal cross sectional views of another catheter of the invention with an outer sheath in relaxed and expanded configurations respectively;

FIGS. 53 and 54 are transverse cross sectional of another catheter of the invention with an outer sheath in relaxed and expanded configurations respectively;

FIG. 55 is an elevational view of a distal end portion of a catheter sheath of the invention;

FIG. 56 is an elevational view of a distal end portion of another catheter sheath of the invention;

FIG. 57 is an elevational view of a distal end portion of a further catheter sheath of the invention;

FIGS. 58 and 59 are elevational views of a distal end portion of another catheter sheath in the retracted and expanded configurations respectively;

FIG. 60 is an elevational view of a distal end portion of a further catheter sheath incorporating radio opaque markers;

FIGS. 61 to 64 are x-ray views of the markers at 0°, 90°, 180°, and 270° respectively;

FIGS. 65 and 66 are elevational views of a distal end portion of another catheter sheath with a collar to direct fluid proximally;

FIGS. 67 to 84 illustrate the use of the invention in a balloon angioplasty procedure;

FIG. 85 is a perspective view of a guide wire according to the invention;

FIG. 86 is an enlarged view of a distal end of a sheath over portion of the guide wire of FIG. 85 in a retracted configuration;

FIG. 87 is an enlarged view similar to FIG. 86 with the sheath is an expanded configuration;

FIG. 88 is a cross sectional view of another catheter of the invention in one configuration in which the sheath is eccentric in the collapsed configuration;

FIG. 89 is a cross sectional view of another catheter of the invention in FIG. 88 in which the sheath is eccentric in the expanded configuration;

FIG. 90 is a view of a sheath of a composite construction, in this case with a braid within the sheath; and

FIG. 91 is a graph of the degree of reinforcement along the length of another catheter according to the invention.

### DETAILED DESCRIPTION

Referring to the drawings there is illustrated a range of catheters 1 comprising a shaft 2 extending from a proximal end 3 to a distal end 4 and a sheath 5 which extends along the length of the catheter shaft. The sheath 5 comprises a compliant material and is expansile for fluid delivery from a distal end 6 of the sheath 5 to the proximal end 3 of the catheter. The expandable tubular sheath 5 lies flush to, and concentric with, the outer surface of a catheter shaft 2 during delivery and retrieval from the body. The catheter may be advanced over a guide wire 10 in either an over the wire or a rapid exchange configuration. The inner diameter of the sheath 5 is substantially the same as the outer diameter of the shaft 2.

The sheath 5 is mounted to the shaft 2 at the distal end of the catheter. A distal tip of the sheath 5 is secured to the catheter such that it does not dislocate upon insertion through the introducer sheath, upon navigation through the vasculature and/or during delivery of fluid.

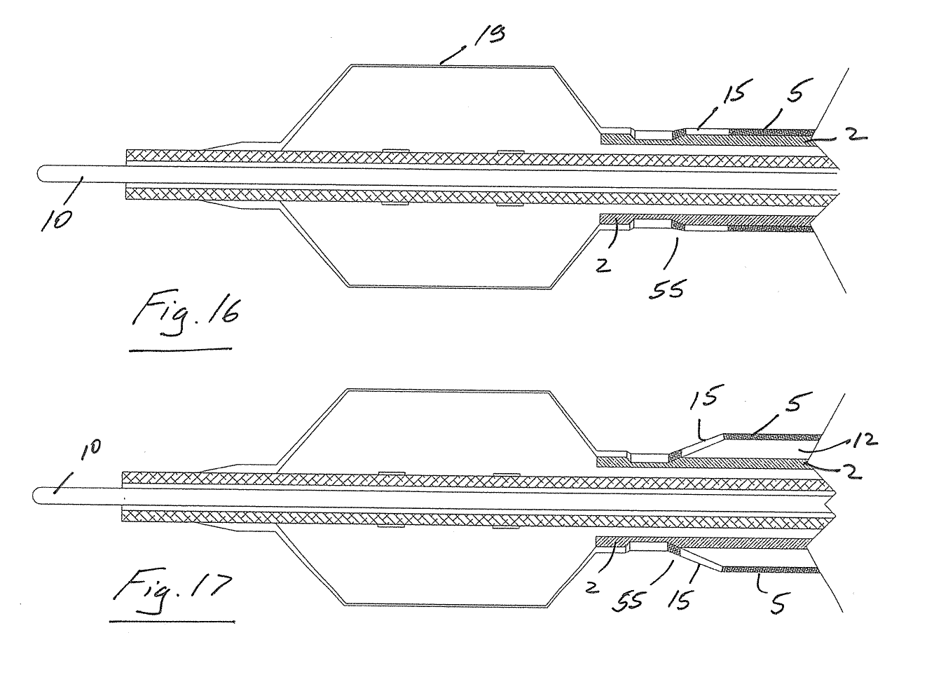

One tip attachment configuration is illustrated in FIGS. 14 and 15. In this case a marker band 50 secures the sheath 5 to the catheter shaft 2 and also provides a radio opaque marker to highlight the fluid exit port location.

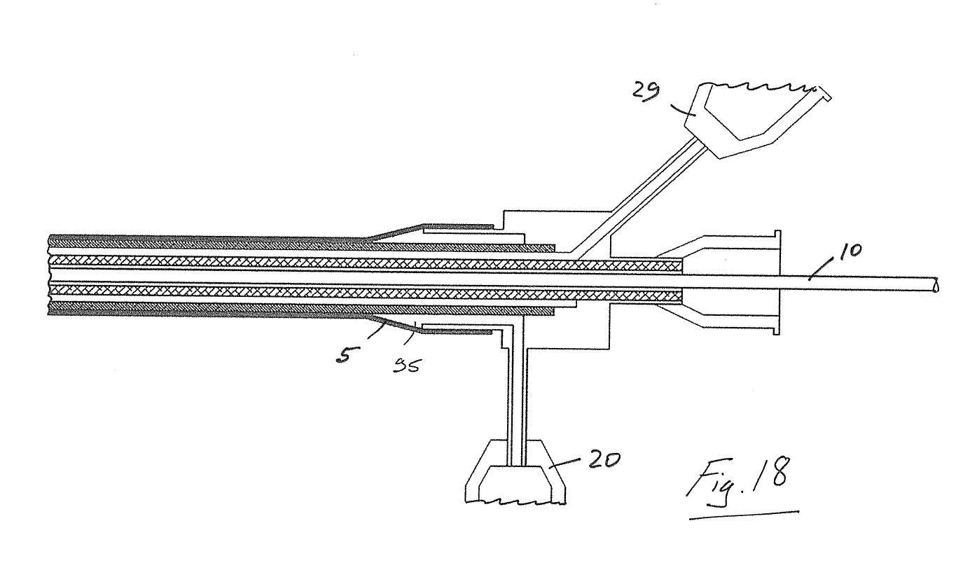

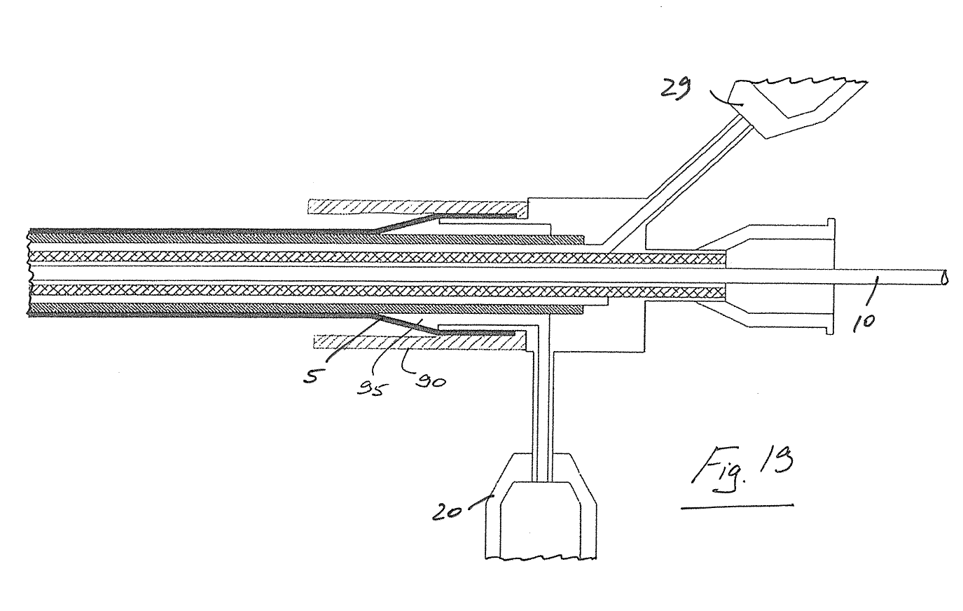

Alternatively or additionally, referring to FIGS. 16 and 17 the distal tip of the sheath 5 may be fused to the catheter using thermal bonding, solvent bonding or adhesive at a joint 55.

The sheath 5 is connected to a Luer fitting 20 at the proximal end of the catheter. The Luer fitting 20 (compared to a conventional catheter) is utilised to provide independent access to the sheath lumen 12 from an inlet port. In the case of a balloon catheter a second Luer is utilised to provide access to the balloon lumen from another inlet port.

In one embodiment the outer surface of the sheath 5 is adhered to the inner surface of the Luer 20 to ensure a sealed connection (FIG. 19).

In one embodiment, to enable fluid to freely pass from the Luer connection 20 there is an increase in the diameter of the sheath at its connection to the Luer 20. This flared geometry provides an in-flow region 95 allowing flow to develop and controlling the initial expansion of the sheath 5 at its proximal end. This reduces the force required to expand the sheath 5, and the potential for localised over-expansion which could lead to the sheath 5 bursting.

In one embodiment, the maximum diameter of the sheath 5, at the connection to the Luer 20, is the same or similar to that of the sheath 5 when in the expanded configuration for fluid delivery. In another embodiment the increase in the diameter of the sheath 5 at the connection to the Luer 20 ensures an open cross-sectional area of the sheath which is at least the cross sectional area of the inlet Luer connection 20.

In another embodiment (FIG. 19 (relaxed), 20 (expanded)) a protecting sleeve 90 is incorporated on the outer surface of the Luer 20 and sheath connection, to prevent over-expansion of the sheath 5 at its proximal end, thus preventing the potential for sheath to burst. Upon expansion of the sheath during fluid delivery this protective sleeve 90 acts as a limiter on the diameter, thus preventing over expansion. This protective sleeve may be comprised of a heat shrink material such as PTFE, PEBAX, FEP.

In another embodiment (FIG. 21), the outer surface of the sheath 5 is connected to inner surface of a support tube 97. The outer diameter of this support tube 98 is connected to the inner diameter of the Luer 20. This enables ease of manufacture as the sheath 5 can be held by the support tube 97 during manufacture and when the sheath 5 is pulled on over the shaft.

Upon injection of fluid/particles through the Luer 20, the sheath 5 comprising the compliant material expands to provide a temporary lumen 12 which facilitates flow of fluid/particles to flow from the proximal end 3 of the catheter to an opening 15 or openings 15 adjacent to the distal end 6 of the sheath 5 and at or near the distal end 4 of the catheter shaft 2 for delivery of the fluid/particles into a vessel.

Upon cessation of injection pressure, the sheath 5 contracts immediately and automatically to provide a lower profile to easily accommodate repositioning/removal of the catheter. On automatic contraction of the sheath 5, the temporary lumen 12 is no longer provided and the profile of the shaft/sheath is minimised.

The expandable sheath 5 comprises a compliant material and is sufficiently expandable under low pressure such as to allow the required volume of fluid (typically less than 20 ml) through the lumen 12 in a short period of time (typically less than 20 seconds). The sheath 5 automatically recovers to its original low profile once pressure is removed. The sheath 5 operates with a thin wall thickness (typically approx. <0.01 inch) to ensure that the outer profile of the catheter is not compromised. The sheath 5 also withstands the frictional forces acting on it during delivery and retrieval. The syringe pressure (or force) required to expand the sheath 5 to create the temporary lumen 12 for fluid delivery does not exceed that which is used in conventional clinical practice. Because the sheath 5 comprises a compliant material it returns automatically to the low profile delivery configuration.

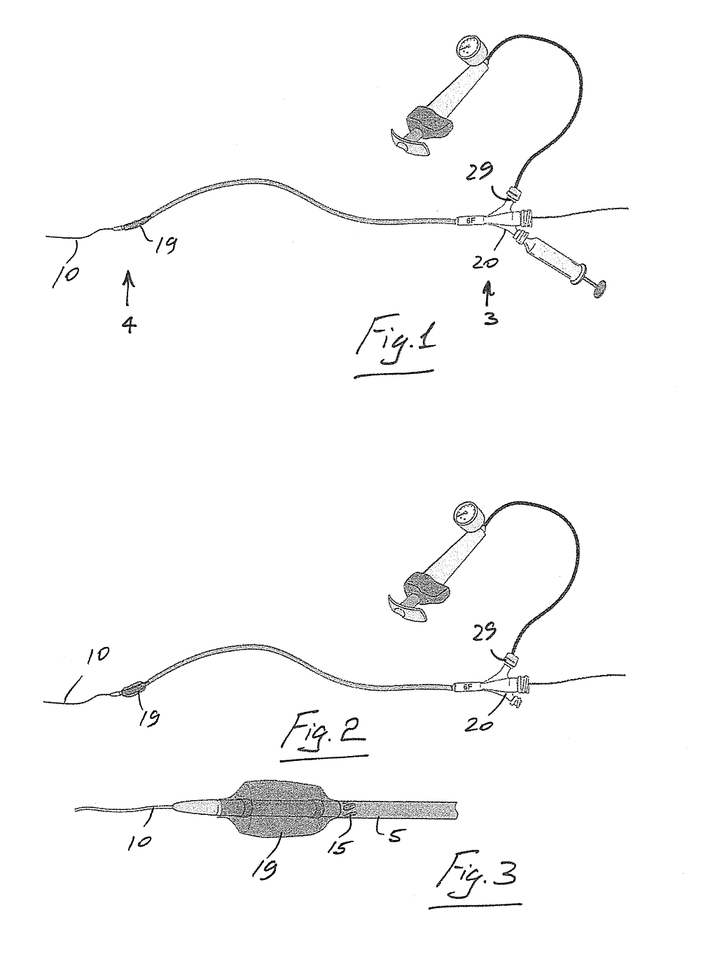

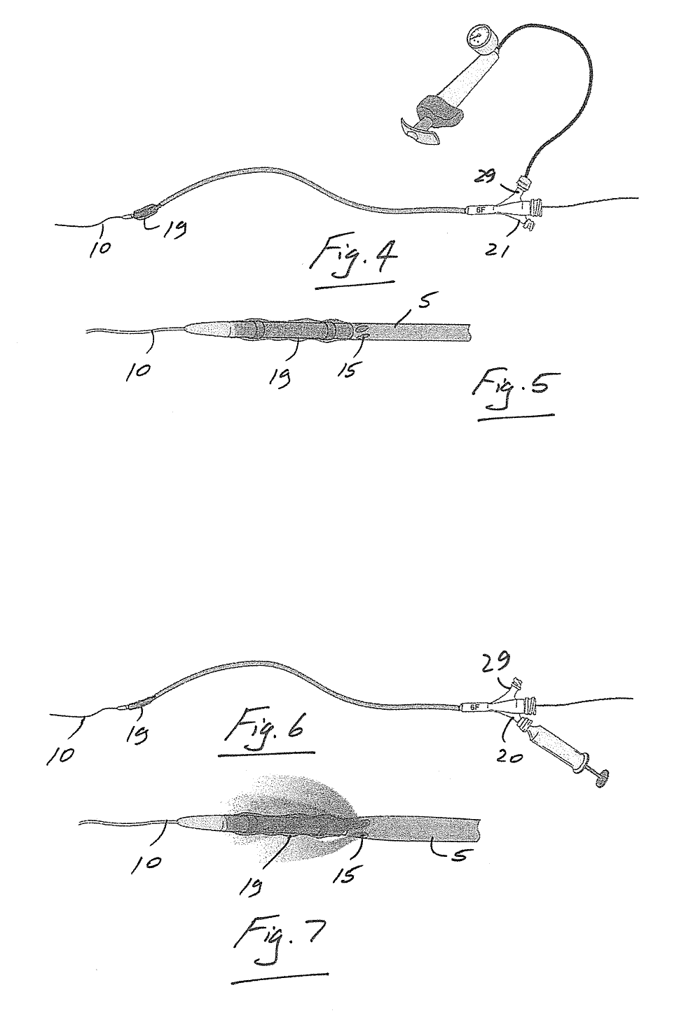

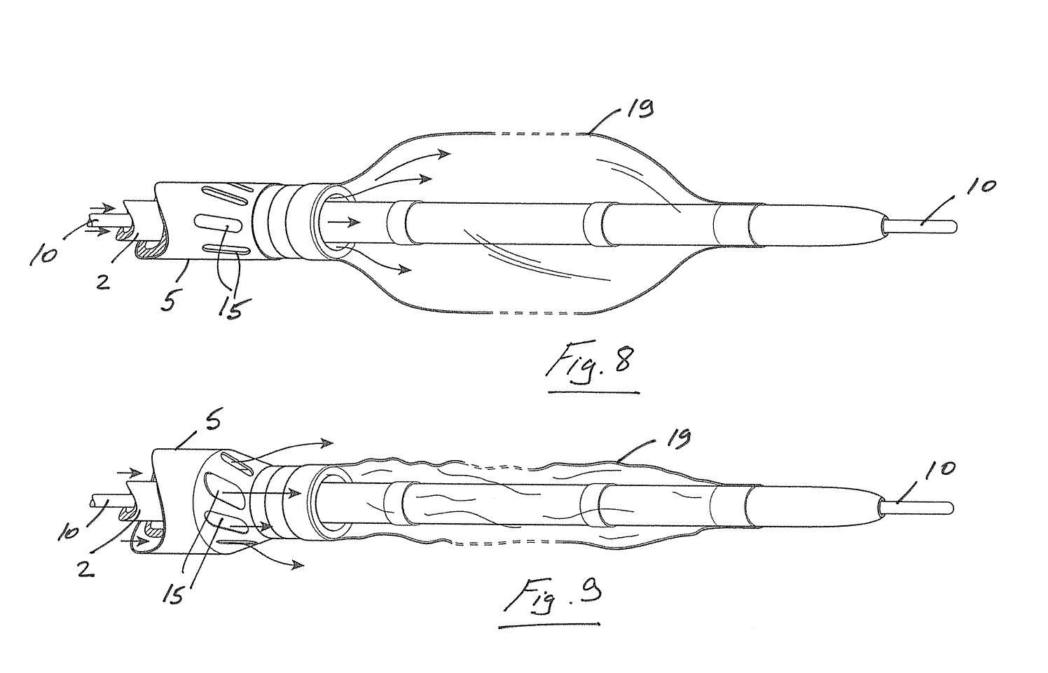

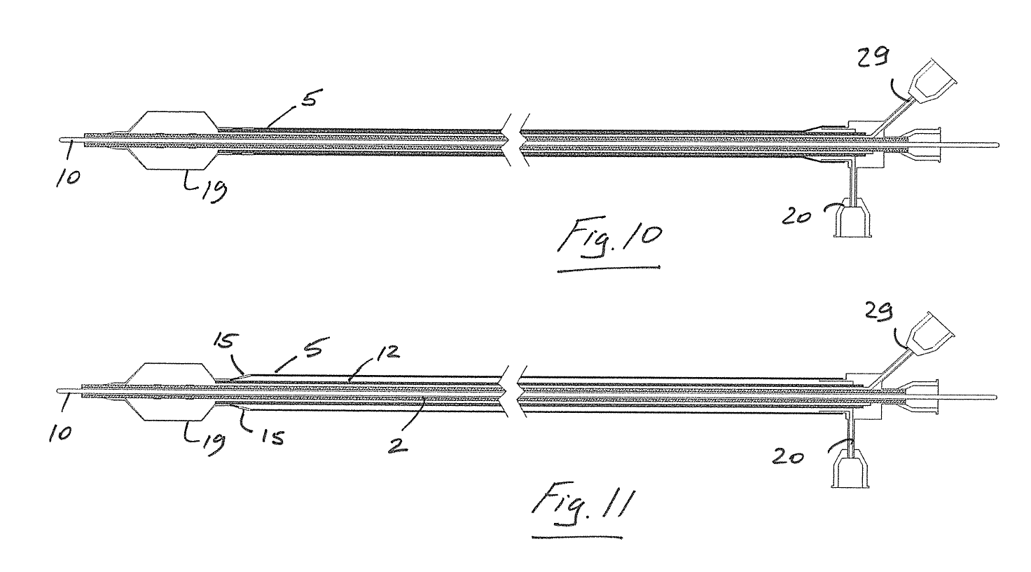

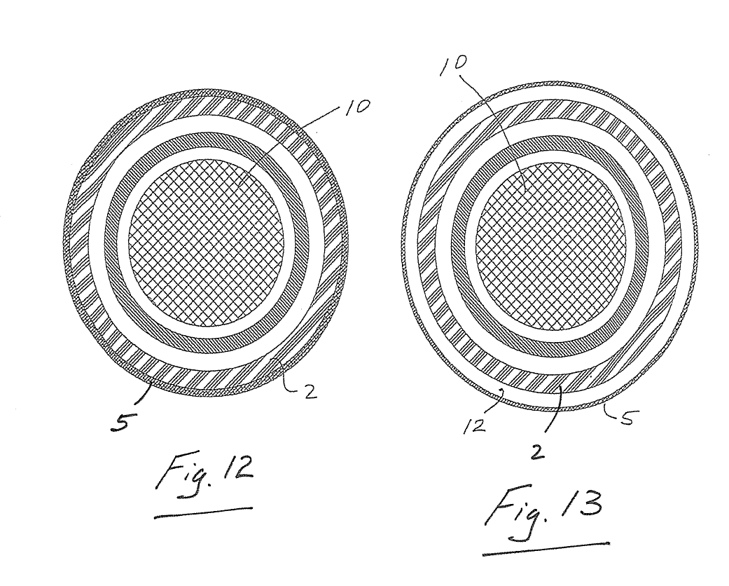

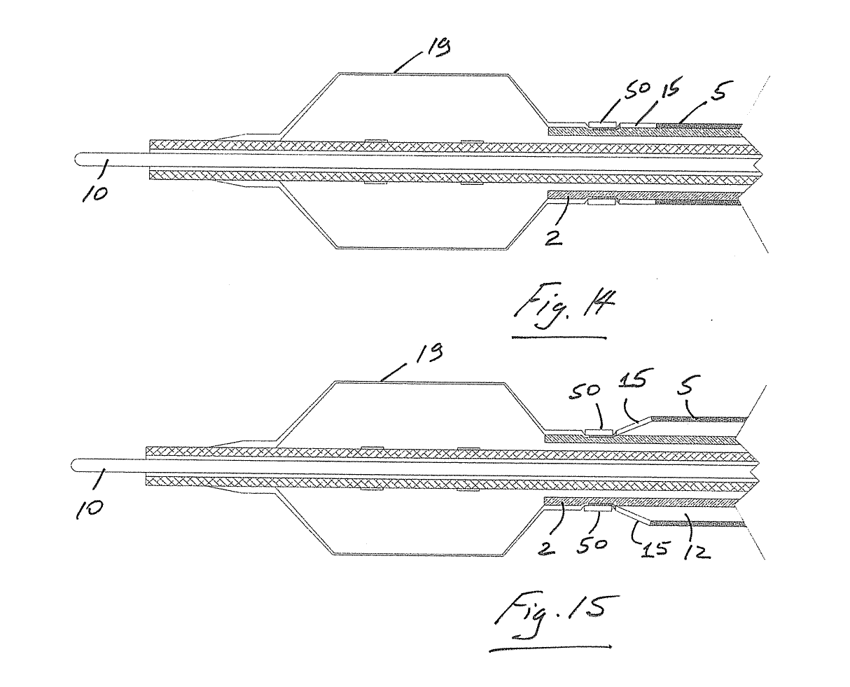

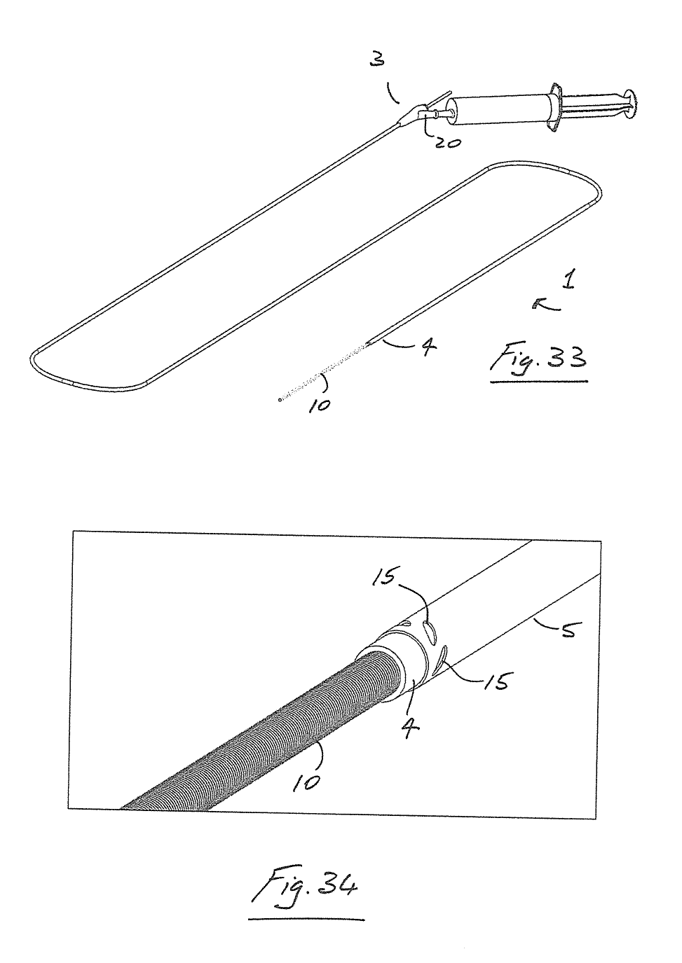

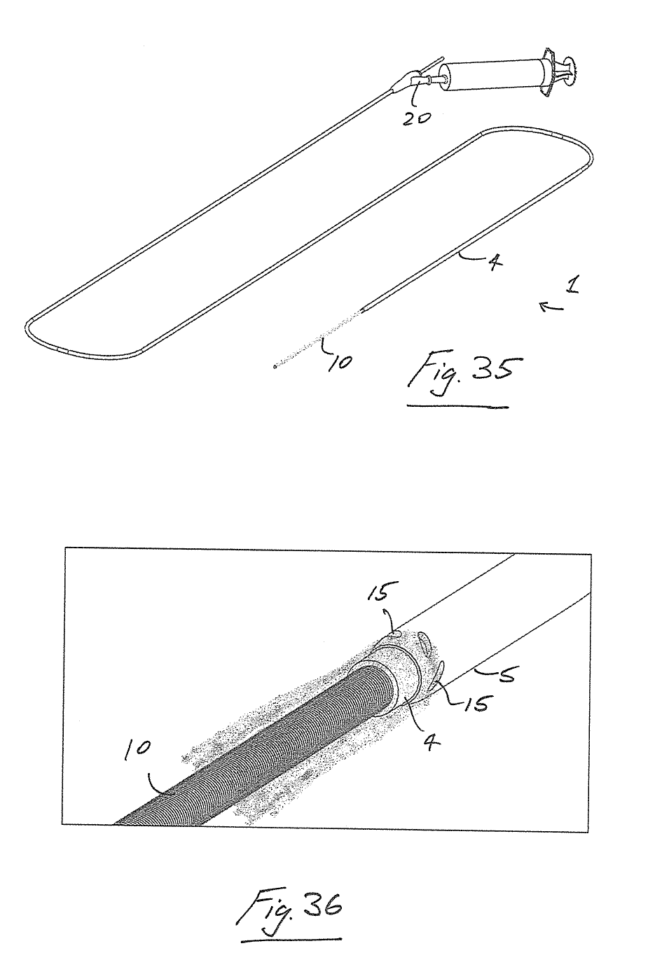

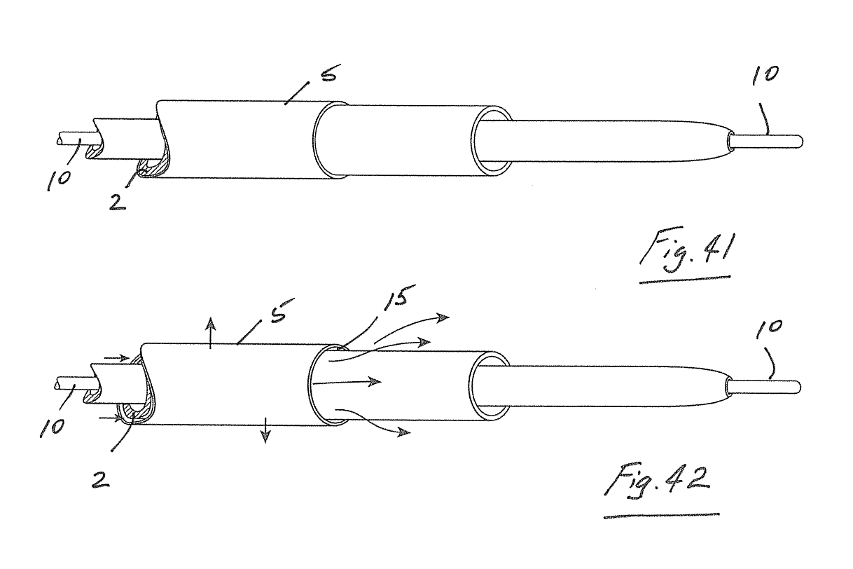

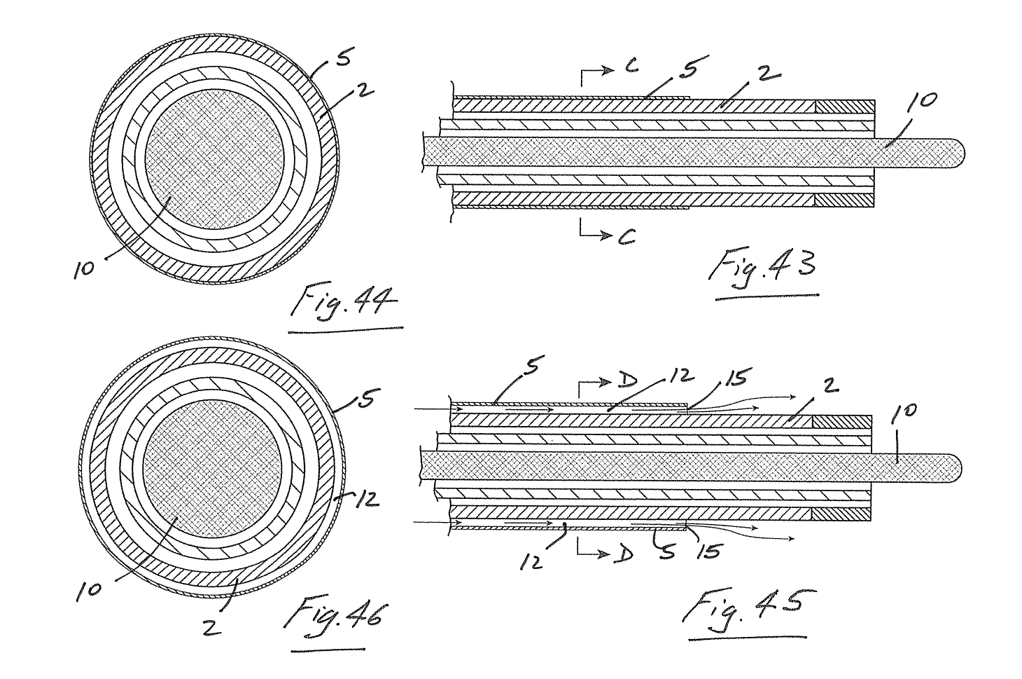

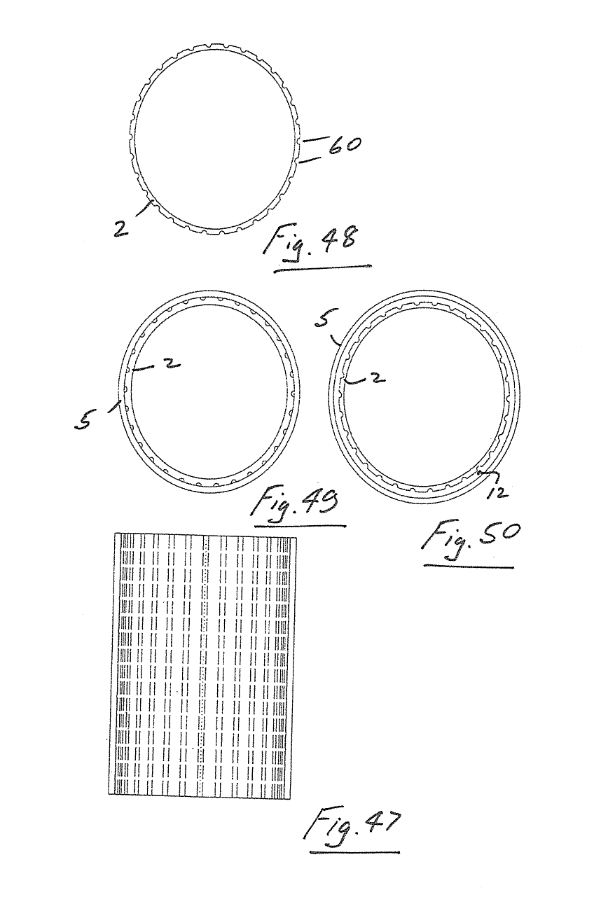

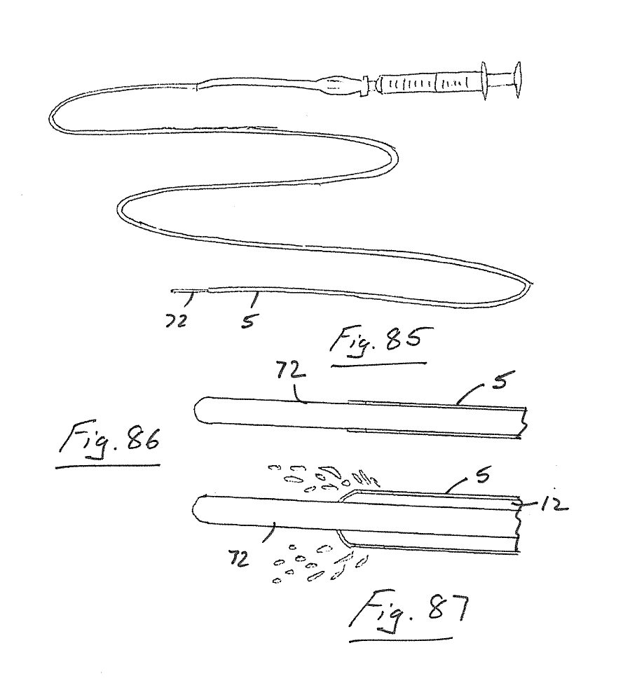

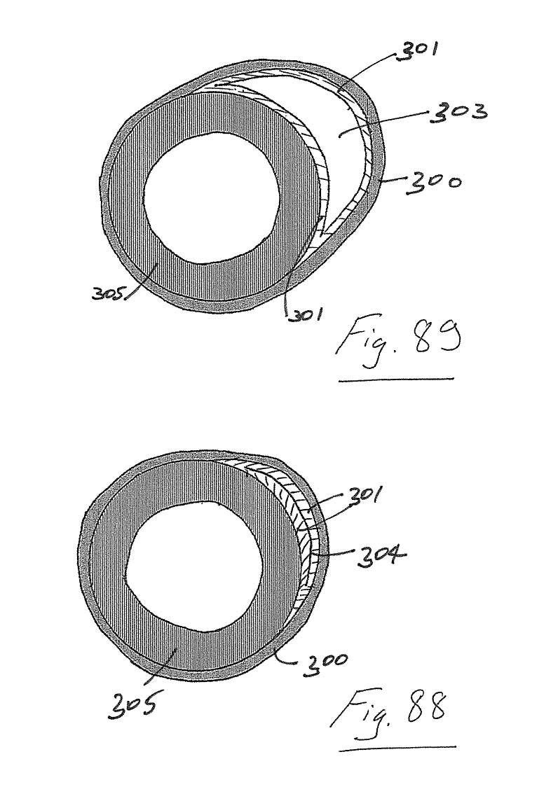

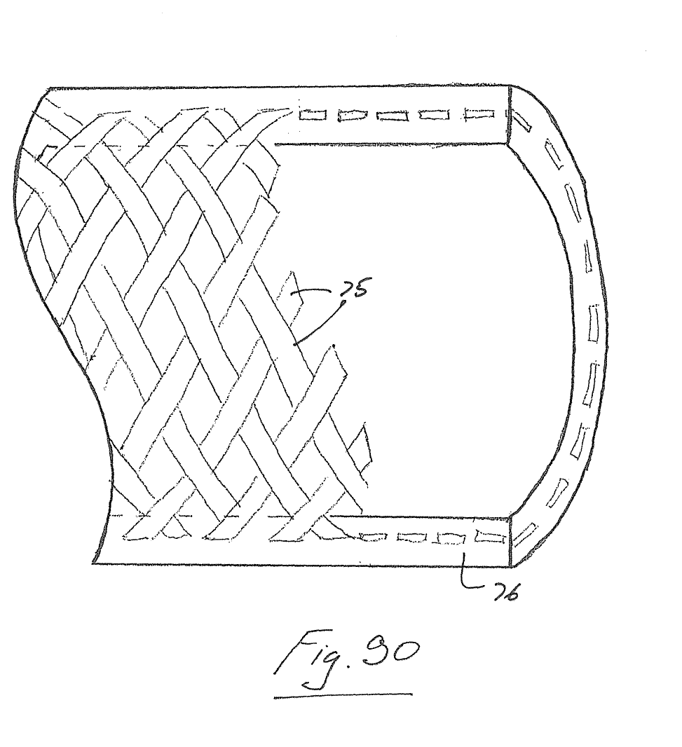

The catheter may for example comprise a guide wire (for example FIGS. 85 to 87), a guide catheter (for example FIGS. 33 to 46) or a balloon catheter (for example FIGS. 1 to 18). A balloon catheter incorporates a conventional distal balloon 19.

Referring to FIGS. 17 and 18, in this case the external expandable sheath 5 is formed from a compliant material which expands when fluid is injected into the Luer 20 and recovers to automatically collapse the lumen 12 to the lower profile when the injection pressure is reduced/removed.

The sheath 5 may comprise Silicone, Polyurethane, Pebax, Polyurethane, Polydimethylsiloxane, Polyolefin, Polypropylene or other compliant polymers. Such materials will fit tightly to the outside surface of the base catheter or guide wire during delivery and readily expand under applied pressure to allow fluid transfer from the proximal end to the distal end. The wall thickness of the sheath will be ideally a minimum of 0.0005 in in and a maximum of 0.007 in. In one embodiment the wall thickness may be 0.003 in.

It is preferable to minimise the wall thickness of each of the layers to be as low as possible. This ensures a low profile catheter and prevents the need to apply excessive force to the syringe during fluid injection. For example, a single layer polyurethane sheath of approximately 0.004 in wall thickness applied on a shaft of 0.022 in outside diameter of length 80 cm requires approximately 9 atm of hand pressure using a 2 ml syringe. Using this system a 2 ml injection is delivered in approximately 2 seconds. This is in the range of 0.014 in and 0.020 internal diameter catheters currently on the market (5-10 atm). It is therefore preferable not to increase this wall thickness beyond 0.007 in due to the need for increased pressure for fluid delivery.

Cross linking of the polymer chains of the compliant material may also be utilised to improve the over-expansion resistance. This will ensure that for a given injection pressure the diameter of the sheath will not exceed a desired limit.

In one embodiment the compliant polymer may be combined with nano fillers (additives with at least one dimension in the nano scale) which provide strength to the compliant material to prevent over expansion while still retaining sufficient compliance to permit fluid flow.

In another embodiment a short or long fibre reinforcement may be used to prevent over expansion while retaining the compliant nature of the base material to permit expansion for fluid flow.

In another embodiment the sheath may comprise a braid 75 impregnated with, or surrounded by, a polymer matrix 76, thus remaining a single layer of composite construction (FIG. 90). The braid may comprise a braided wire. In this case a complaint polymer is reflowed into apertures between and around braid wires (wire diameter of 0.0005-0.006) to provide the fluid seal, creating a single layer sheath of two materials. The braid may be metallic (e.g. Nitinol, Stainless Steel, Cobalt Chromium, Platinum, Titanium, Paliney) or polymer and the filler/seal a polymeric material. The braid design ensures that the sheath is limited to a predetermined diameter to prevent over expansion. The total sheath wall thickness is a minimum of 0.0005 in and a maximum of 0.0007 in, and preferably 0.0003 in.

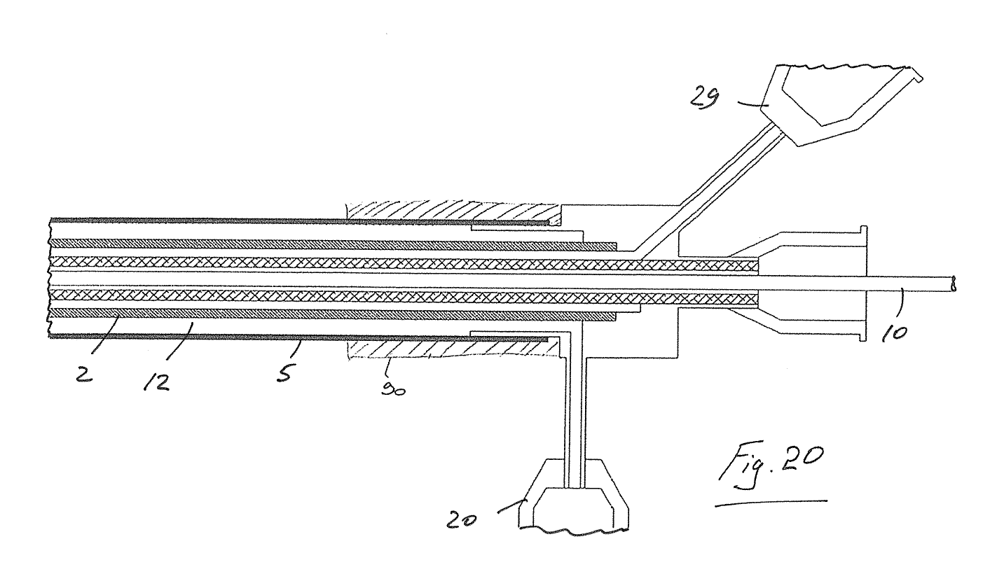

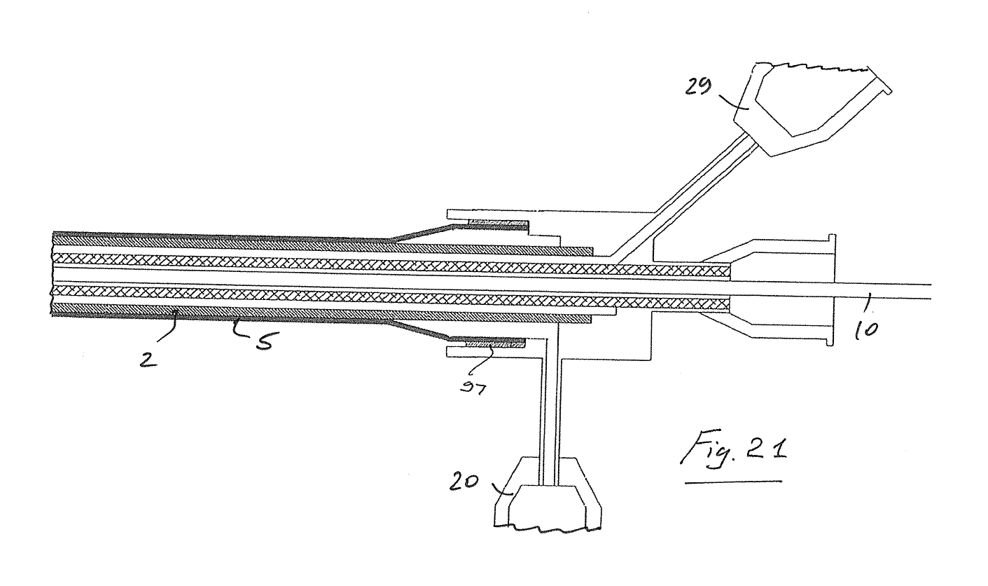

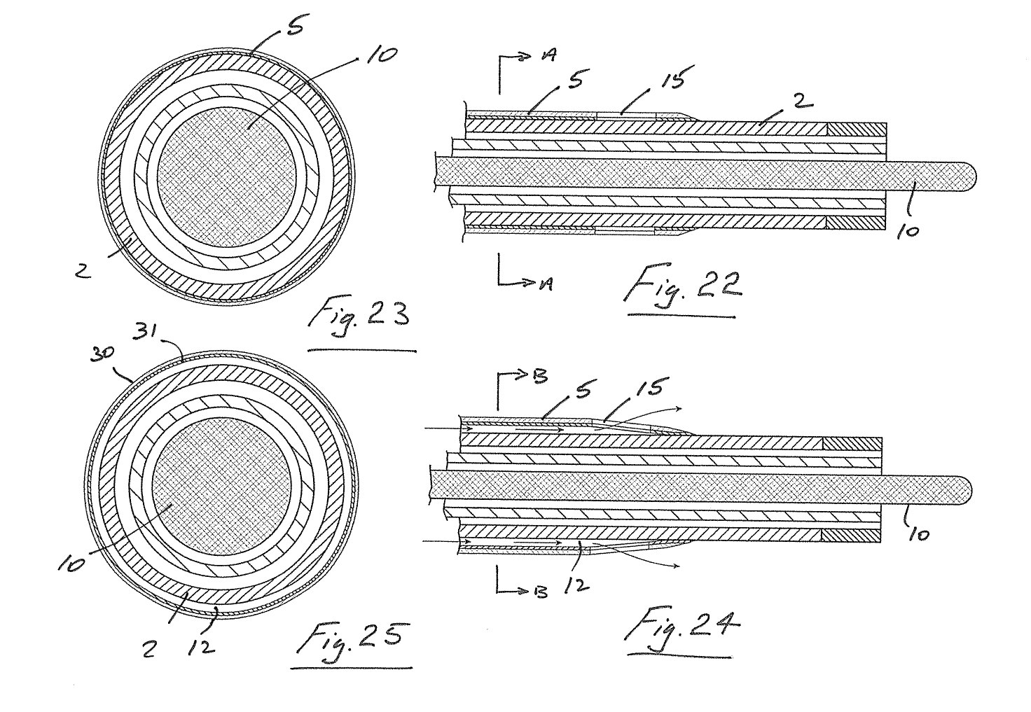

Referring to FIGS. 22 to 28 in this case the external sheath 5 is formed from a compliant (elastic) material 30 and a diameter controlling material 31 combination is utilised. In this configuration the diameter controlling material 31 provides increased burst pressure, resistance to over-expansion and deformation, and/or controls the shape of the sheath under pressure whilst also permitting controlled expansion. This ensures that the outer diameter of the jacket 5 will not exceed a threshold value during fluid injection. In some cases the material which controls the diameter may be porous and the compliant material provides a fluid seal and also helps to force the diameter controlling material to recover once pressure is removed. A third layer may be added inside the diameter controlling material 31 to seal it from fluid.

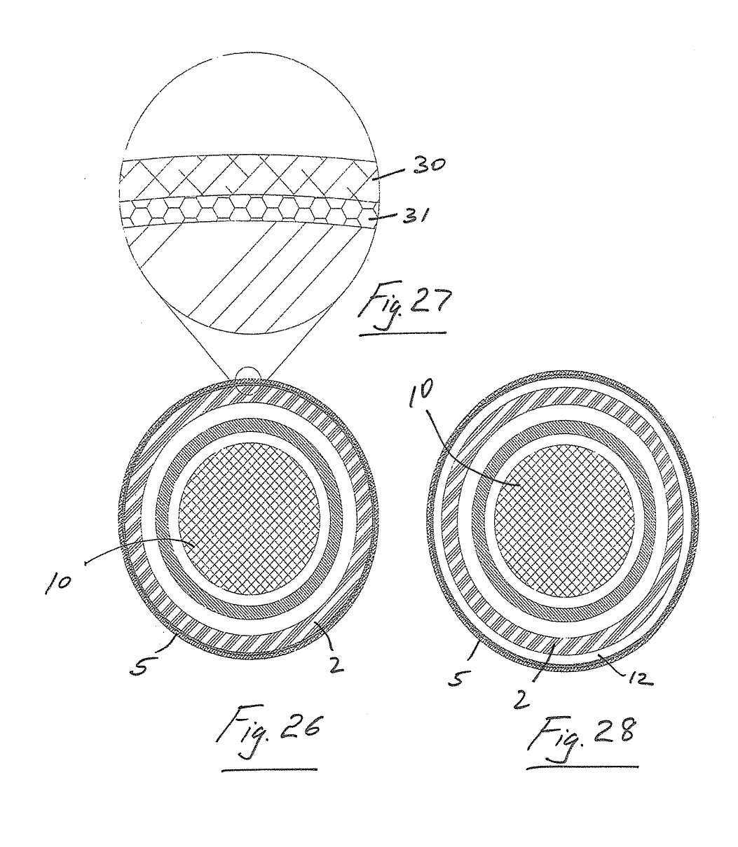

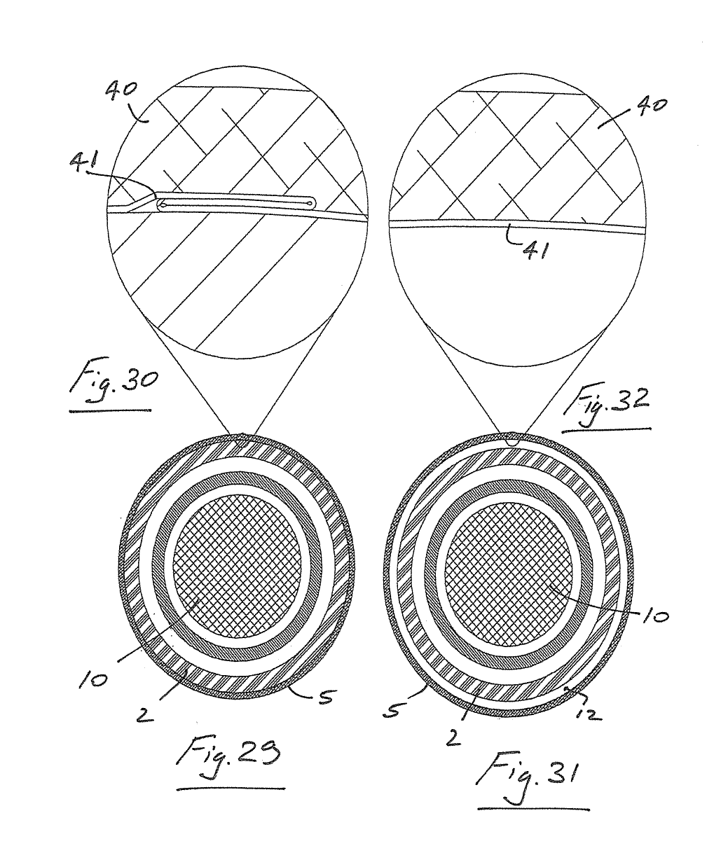

Referring to FIGS. 29 to 32 in this case the external sheath 5 is formed from combination of a compliant material 40 and a non-compliant material 41. In this case at least two layers are required. In this configuration the non-compliant material 41 acts as a reinforcing layer and provides increased burst pressure, resistance to over-expansion and deformation, and/or controls the shape of the sheath under pressure whilst also permitting controlled expansion. The compliant material 40 provides a means to force the non compliant material 41 to recover onto the outer surface of the catheter when pressure is removed.

The reinforcing layer 41 may comprise a suitable non-compliant material such as PET, Polyamide, Polyimide, Pebax, PEEK, or Polypropylene. A stiff elastomer may also be appropriate. A layer of these materials will provide the strength to stop over expansion. This layer would be wrapped onto the outer surface of the catheter and unfolds from a collapsed configuration to allow fluid flow upon fluid injection. When pressure drops this layer would be forced back to a compact profile by the compliant layer. The diameter of the non-compliant layer in the unfolded configuration is equal to the desired maximum diameter of the sheath when in operation. The wall thickness of the non-compliant layer should be a minimum of 0.00025 in and a maximum of 0.004 in. In one embodiment the non-compliant layer wall thickness is 0.0001 in diameter.

The non-compliant layer may comprise a polymer or metallic braid. A braid wire diameter of minimum of 0.00001 in and maximum of 0.005 in and preferably 0.0001 is appropriate. The braid may be metallic (e.g. Nitinol, Stainless Steel, Cobalt Chromium, Platinum, Titanium, Paliney) or polymeric (e.g. PEEK, PET, PP, Polyamide, Pebax). The compliant layer may be comprised of Silicone, Polyurethane, Pebax, Polyetherurethane, Polydimethylsiloxane, Polyolefin, or Polypropylene. The wall thickness of this layer should be 0.0007 to 0.004 in. In one embodiment the wall thickness is 0.0001 in.

The sheath may comprise three layers; a reinforcing layer, a compliant layer, and a sealing layer. In this case the reinforcing layer and compliant layers may be porous, and do not provide a fluid seal. These may be of a braid or other porous construction.

Polymeric materials can adhere to one another over time, due to elevated temperature during sterilisation and storage, or even due to the molecular structure of the materials themselves. This could result in increased force to expand the jacket and deliver the fluid.

A number of methods to ensure effective operation of the jacket expansion are outlined below.

Application of lubricious coatings which are readily available such as hydrophilic, hydrophobic, silicone and the like. These may be applied to one or both of the outer surface of the catheter and the inner surface of the jacket to prevent adhesion.

Alternatively or additionally the surface texture of the outer surface of the catheter may be modified.

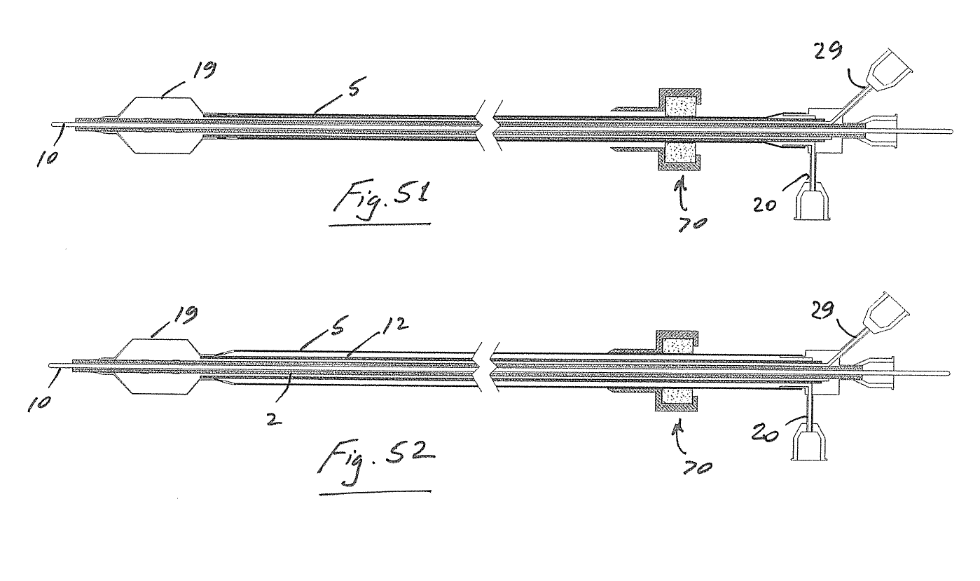

Referring to FIGS. 47 to 50 indentations or striations 60 may be formed on the outer surface along at least portion of the length of the catheter shaft 2. This will lower the adhesion force by lowering the surface area of contact between the outer sheath 5 and the inner shaft 2 thereby reducing the force. In addition, multiple small channels are created for the fluid to flow under the sheath 5 thus creating a larger surface area for the fluid to act on expanding the jacket as illustrated in FIG. 50.

The catheter can be used with connection introducers, the hemostasis valve on the introducer may require a dilation force in excess of the force deliverable through the expandable lumen.

The catheter may include a means of mitigating the risk of the introducer haemostasis valve creating a pinch point on the jacket thus restricting fluid flow.

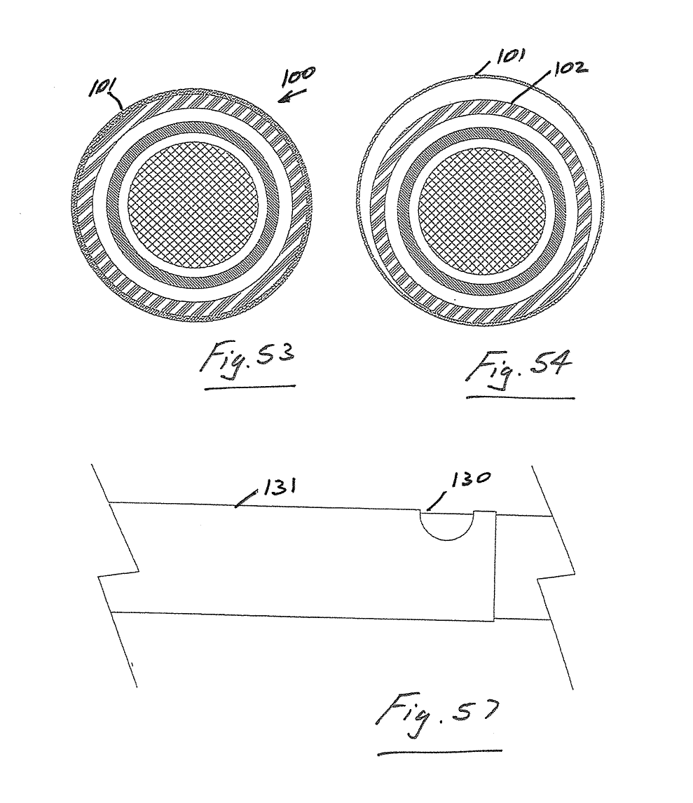

For example, referring to FIGS. 51 and 52 a slider valve engages 70 with the haemostasis valve on the introducer sheath which effectively deactivates the valve. A separate valve is provided on the slider which is customised to ensure that it restricts fluid flow along the length of the catheter (i.e. blood escaping from the vasculature) but is readily expandable radially to permit full function of the expandable sheath under low pressure.

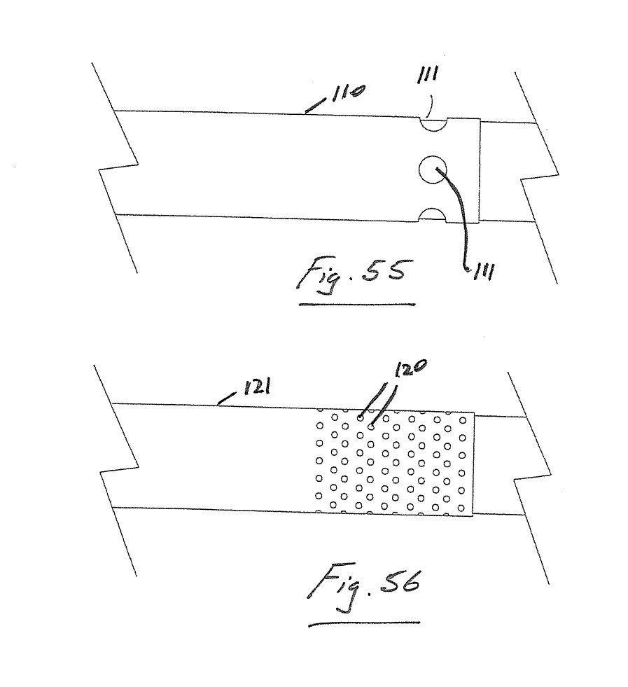

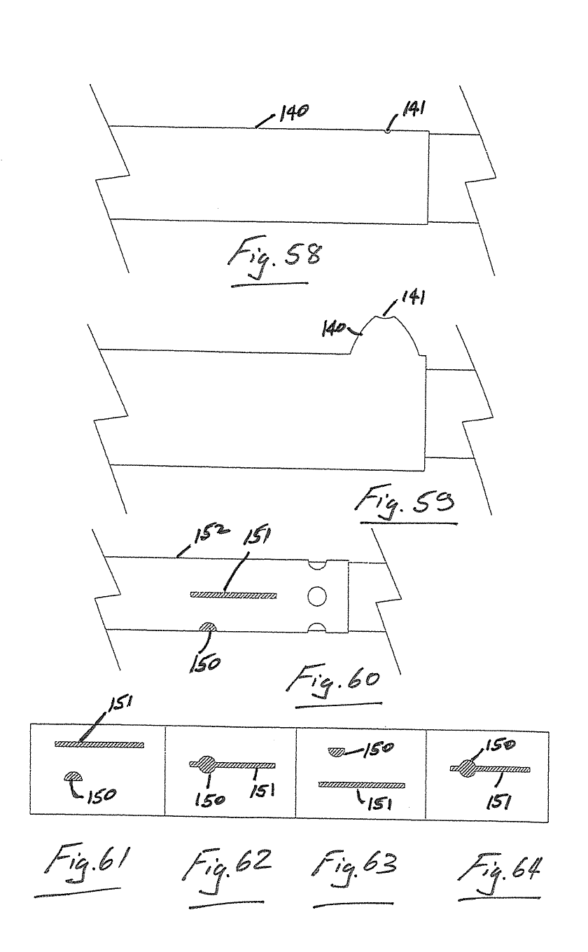

Referring to FIGS. 53 and 54 there is illustrated another catheter 100 of the invention in which the outer sheath or jacket 101 is secured to only portion of the outer surface of the catheter shaft 102. This promotes non-concentric sheath expansion as illustrated in FIG. 54.

Referring to FIG. 55 there is illustrated a sheath 110 which in this case has a number of outlets 111 adjacent to the distal end thereof.

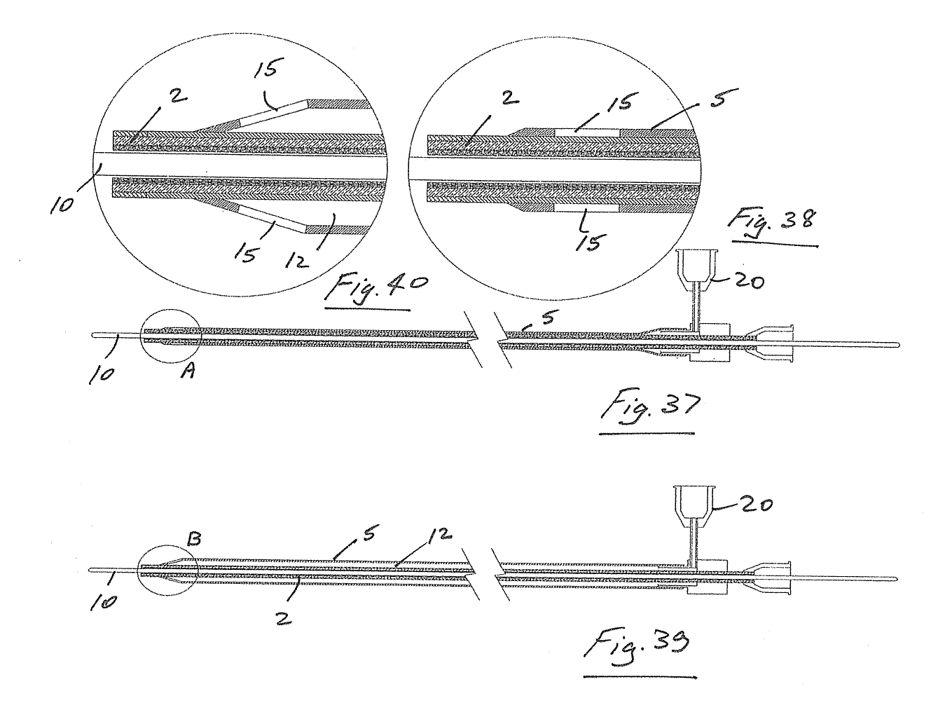

In one case there are multiple outlet holes 120 in an outer sheath 121 as illustrated in FIG. 39.

Referring to FIG. 57 there may be a single outlet hole 130 in an outer sheath 131.

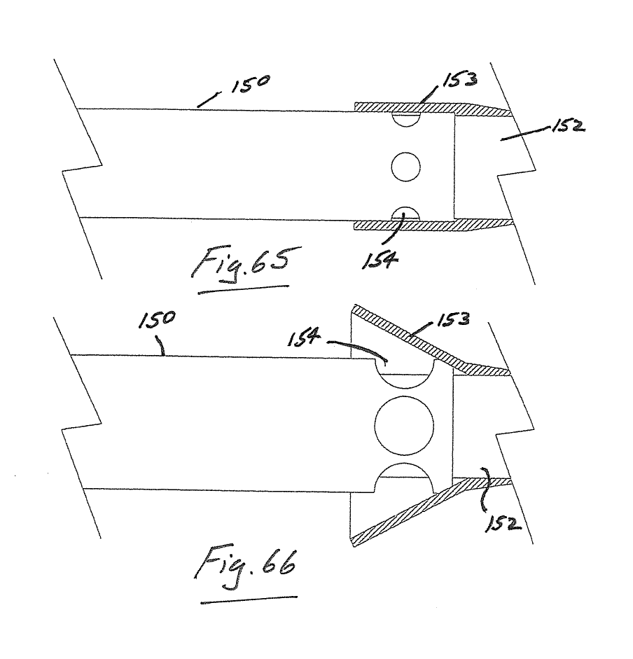

Referring to FIGS. 58 and 59 a sheath 140 may be inflatable only local to an outlet hole 141 in order to direct fluid to a target location. Such an arrangement facilitates accurate delivery to, for example, a side branch vessel.

Referring to FIGS. 60 to 64 radio opaque markers 150, 151 may be incorporated with a sheath 152 to define rotational orientation of the catheter. FIGS. 61 to 64 are x-ray views at 0°, 90°, 180° and 270° respectively.

Referring to FIGS. 65 and 66 sheath 150 and associated catheter shaft 152 are provided with a collar 153 which extends over sheath exit ports 154 as illustrated. In this case the collar is configured to expand on inflation of the sheath 150 to direct fluid exiting from the exit holes 154 proximally.

In the catheter of the invention the lumen of the sheath is concentric with the outer lumen of the catheter until pressurised. This provides for uniform flexibility in all directions. This ensures that the catheters trackability is not affected and the shaft does not twist when traversing tortuous anatomy (i.e. parallel tube configuration is more flexible in one plane than another and so can twist to find the ‘path of least resistance’ when pushed through a bend).

The lumen is collapsed onto the outer surface of the catheter until pressurised. This maintains a low profile (adds approx. 0.1-0.5Fr to the diameter) with the outer surface of the catheter until pressurised. This allows for crossing narrow lesions and small diameter vessels.

The catheter of the invention has an expandable lumen travelling full length of catheter. This ensures that the full length of the catheter is low profile when no fluid is being delivered.

In the invention there is a separate fluid delivery lumen with an independent Luer. No need to remove guide wire/catheter prior to delivering fluid, thereby reducing patient risk, procedural time, radiation exposure and contrast dose. The user has full control over balloon inflation/deflation even while delivering fluid

The catheter of the invention may be used in a wide range of applications including:—
balloon angioplasty for widening narrowed or obstructed blood vessels;
blood vessel stenting (both balloon expandable and self expanding stents) procedures to scaffold a localised restriction in a vessel;
diagnostic procedures using guiding catheters/sheaths;
any drug/therapeutic/embolic/biologic agent delivery involving catheters;
clot aspiration/retrieval procedures to remove unwanted blood clots from arteries/veins; catheters for antegrade/retrograde delivery of cardioplegia;
ablation catheters—radio-frequency, thermal and the like; and
procedures which require two or more fluids to be mixed together within the body.

The technology provides for a separate delivery lumen without substantially increasing the profile of the catheter when the lumen is not in use.

In another embodiment, an expandable sheath 5 is placed on a guide wire 72 which functions similar to a catheter shaft as described above. This is shown schematically in FIGS. 85 to 87. This aspect enables the delivery of fluids along the guide wire, without the need for a catheter. This will enable a substantially reduced profile device for fluid delivery, and enhanced flexibility.

In this case a guide wire 72 extends from a proximal end to a distal end and a sheath 5 which extends substantially along the full length of the wire 72, the sheath 5 having a proximal end and a distal end and being expansile for fluid delivery from the proximal end of the sheath to the distal end of the sheath. The proximal end of the wire will have a means for connection of a syringe or other device for fluid delivery. The distal end of the sheath 5 may be attached to the distal end of the wire 72, and have at least one, or a plurality of holes to enable fluid ejection as described above.

The guide wire 72 enables investigation or treatment of smaller more distal vessels, which cannot be accessed easily using catheters. This diagnostic or therapeutic guide wire has extended functionality over existing guide wires which only allow access to vessels, but cannot be used to deliver a fluid.

Upon injection of a fluid through a catheter with an expansile sheath, the haemostasis valve may restrict expansion of the sheath. Furthermore, if the sheath is very thin, comprised of a very elastic material, it may expand only in the region proximal to the valve. As more fluid is injected, the pressure will increase within the sheath proximal to the valve causing it to bulge and perhaps even burst. This will prevent fluid from travelling distal to the hemostasis valve, and to the distal end of the catheter.

A method of reinforcement may be required to alleviate this problem. A reinforcing layer, such as that described for diameter control may also be used to prevent restriction by the hemostasis valve.

In one embodiment the expansile sheath comprises two concentric layers; an elastic layer, which can expand when a fluid is delivered through it, but automatically contracts afterwards, and a reinforcing layer which prevents restriction by the hemostasis valve (and over expansion of the elastic layer). This will also prevent any over-expansion of the expansile sheath beyond a desired diameter.

The two layers may be adhered to one another.

In one embodiment both layers are concentric with the shaft of the catheter.

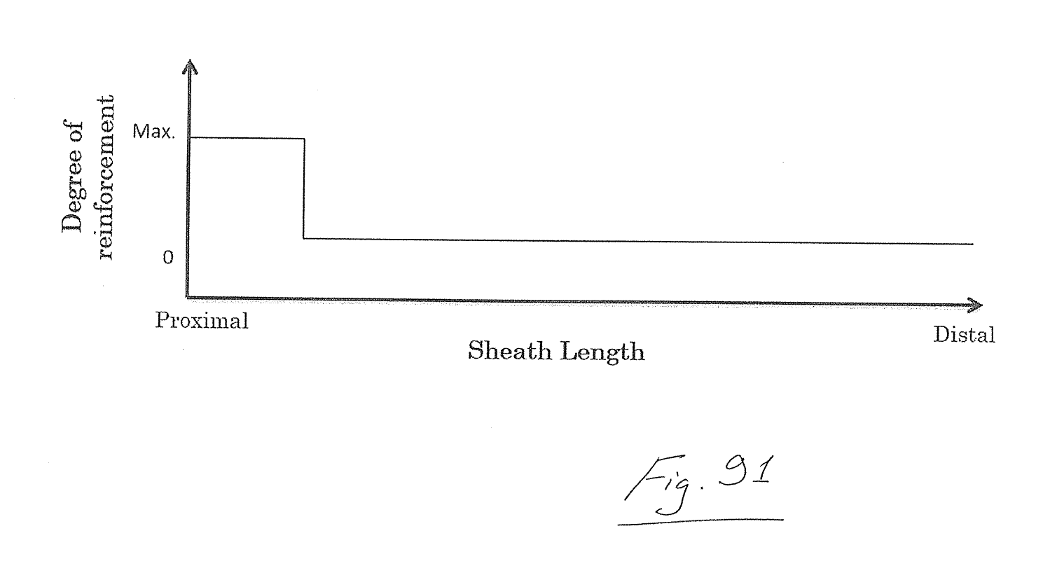

Referring to FIGS. 88 and 89 there is illustrated another catheter of the invention which is similar to those described above. In this case the sheath 5 comprises an outer layer 300 of compliant material and an inner body 301 of non-compliant material. The inner body 301 is laid down on portion of the outside surface of the shaft 305 as illustrated in FIG. 88. The inner body in cross section is somewhat like a bicycle tube in a deflated configuration with a continuous wall 301 in sections on either side of a closed interface 304 in the deflated/relaxed configuration. On inflation, the inner body expands opening up an internal lumen 303 defined by the wall 301. The two layers 300, 301 of the sheath are non-concentric as illustrated in FIGS. 88 and 89. This creates a co-axial lumen 303 for fluid delivery. The perimeter of this lumen 303 is lower than that which would be present in a co-axial lumen of equivalent cross section. This means reduced shear on the fluid as it is injected through the lumen, meaning a lower pressure drop across the catheter length, and consequently a lower force for injection. There is no sacrifice in profile over the approaches described above because the same volume of material is added, regardless of whether the lumens are co-axial or, as in this case, eccentric. One advantage of this embodiment is that it may be relatively easy to manufacture. For example, once the inner body 301 is laid on the shaft 305 the outer compliant layer 300 can be easily pulled over the sub-assembly.

In the embodiment of FIGS. 88 and 89 the sheath is eccentric to the shaft in both the relaxed and the expanded configurations.

In another embodiment, the diameter limiting and reinforcing layer may of a braid type construction, the other layer ensuring that the sheath is sealed. The sealing layer may also be elastic.

Furthermore, the braid may be within the sealing layer, i.e. a composite type structure, as shown in FIG. 90. The braid may be comprised of a polymer, or a metallic material.

A sheath, comprising an elastic material with a braid within may have the propensity to form pinholes during expansion or manipulation. Any porosity in the sheath may be alleviated by impregnation of silicone or an elastomer.

In another embodiment the reinforcing layer may be adhered to the elastic layer.

This reinforcement of the sheath may extend along the entire length of the catheter. However, this may have implications for the flexibility and profile of the catheter.

Increasing the thickness of the sheath, and/or adding a reinforcing layer may increase the profile and stiffness of the catheter. This may not be ideal as for catheters intended to access very tortuous vessels. A lower profile enables better flexibility which is particularly important at the distal end of the catheter.

Typically the physician will insert the catheter a minimum distance into the vessel before delivering any fluid. Accordingly, and to preserve the flexibility of the catheter at the distal end, it may be preferable to only have the reinforcing layer, or increased expansile sheath thickness, along the proximal portion of the catheter as shown in FIG. 91.

In one embodiment it may be preferable to only extend the reinforcing layer from the Luer along 20 cm of the overall catheter length. In another embodiment it may be preferable to only extend the reinforcing layer (or increased wall thickness) from the hub along 30 cm of the overall catheter length. In another embodiment it may be preferable to only extend the reinforcing layer from the hub along 40 cm of the overall catheter length. In another embodiment it may be preferable to only extend the reinforcing layer from the hub along 60 cm of the overall catheter length. In one embodiment it may be preferable to only extend the reinforcing layer from the hub along 80 cm of the overall catheter length

The sheath may also be used as a means of varying the overall flexibility and pushability of the catheter along the length. This may be achieved by varying its properties from the proximal to the distal end. This could be achieved through variations in material, thickness construction, or addition of reinforcing layers as outlined above.

The use of a separate delivery lumen for the sheath provides some or all of the following advantages:
reduction in the number of catheter and guide wire exchanges;
reduction in the risk of losing guide wire position, thereby preventing risks of having to recross/relocate guide wire and/or catheter and reduction in time to reposition same;
reduction in risk involved with removing and reinserting guide wires and catheters into the vasculature;
reduced procedure times due to the reduction in time to recross/relocate devices and fewer procedural steps to complete procedure;
reduced radiation exposure due to reduced procedural times and reduced procedural steps;
reduced contrast load due to reduced procedural times and reduced procedural steps; ability to inject contrast straight to a target site;
reduced sedation time due to reduced procedural times; and/or
reduction in overall procedural cost.

### Current Approach to Delivering Contrast Media (or Other Fluids) Through a Guide Catheter

1. Insert guide wire
2. Advance catheter over guide wire
3. Remove guide wire
4. Place guide wire in saline and heparin bath
5. Deliver contrast media through lumen of catheter
6. Flush lumen with saline
7. Reinsert guide wire
8. Move guide wire into next target vessel
9. Advance catheter over wire
10. Repeat steps 3 to 9 as necessary
   Approach with Current Invention on a Guide Catheter/Angiographic Catheter
1. Insert guide wire
2. Advance catheter over guide wire
3. Deliver contrast media through expandable lumen of catheter
4. Move guide wire into next target vessel
5. Advance catheter over wire
6. Repeat steps 3 to 5 as necessary

### Current Approach to Angioplasty

1. Insert guide wire
2. Advance catheter over guide wire
3. Inflate the balloon
4. Deflate the balloon
5. Remove catheter/guide wire
6. Place catheter/wire in saline and heparin bath
7. Deliver contrast media through introducer sheath/guide wire lumen
8. Flush lumen with saline
9. Reinsert catheter
10. Re inflate balloon
11. Deflate balloon
12. Remove catheter/guide wire
13. Place catheter/wire in saline and heparin bath
14. Deliver contrast media through introducer sheath/guide wire lumen
15. Repeat steps 7 to 13 as necessary
   Approach with Current Invention on a Balloon Catheter

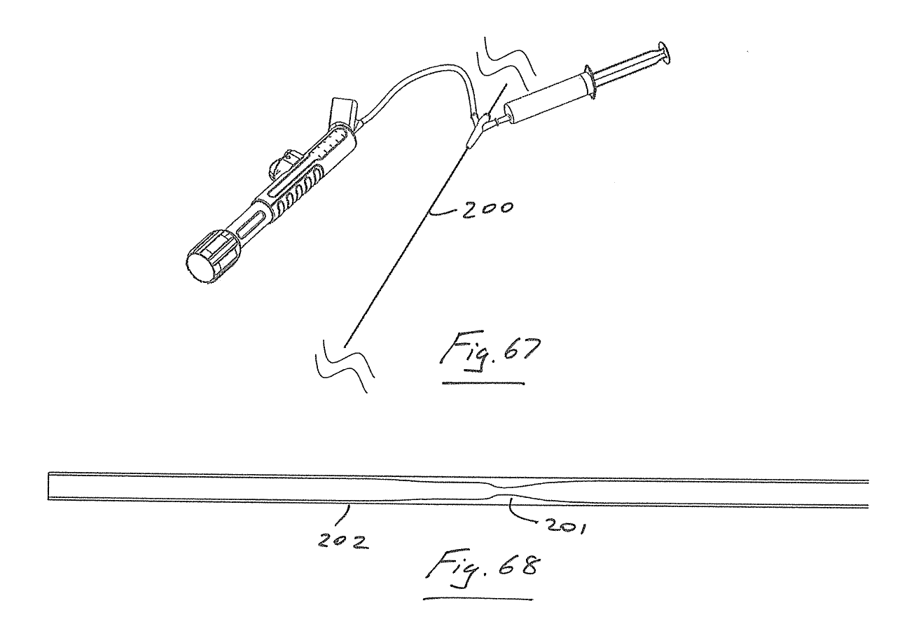

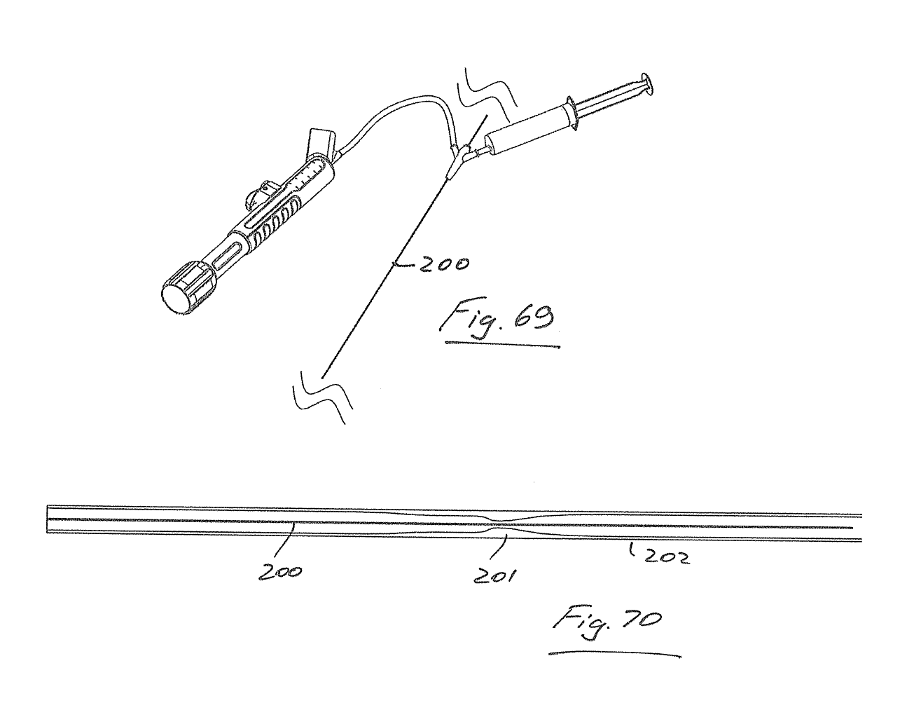

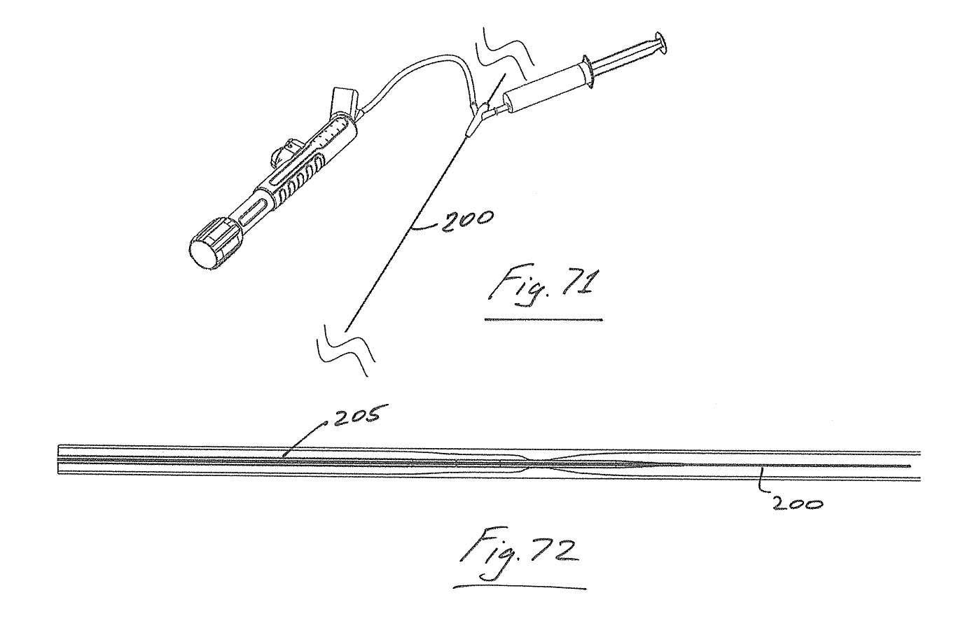

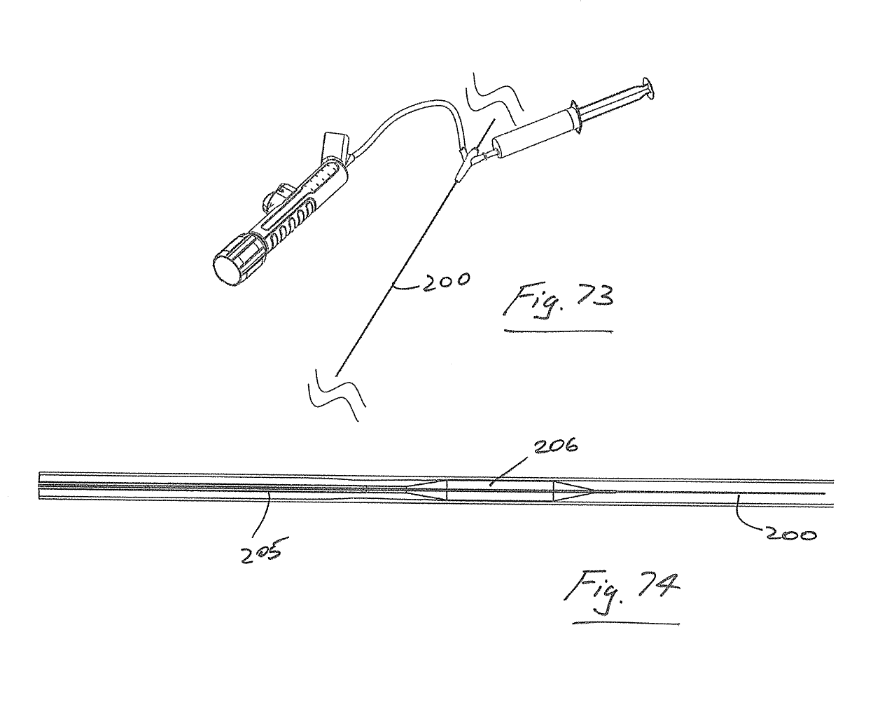

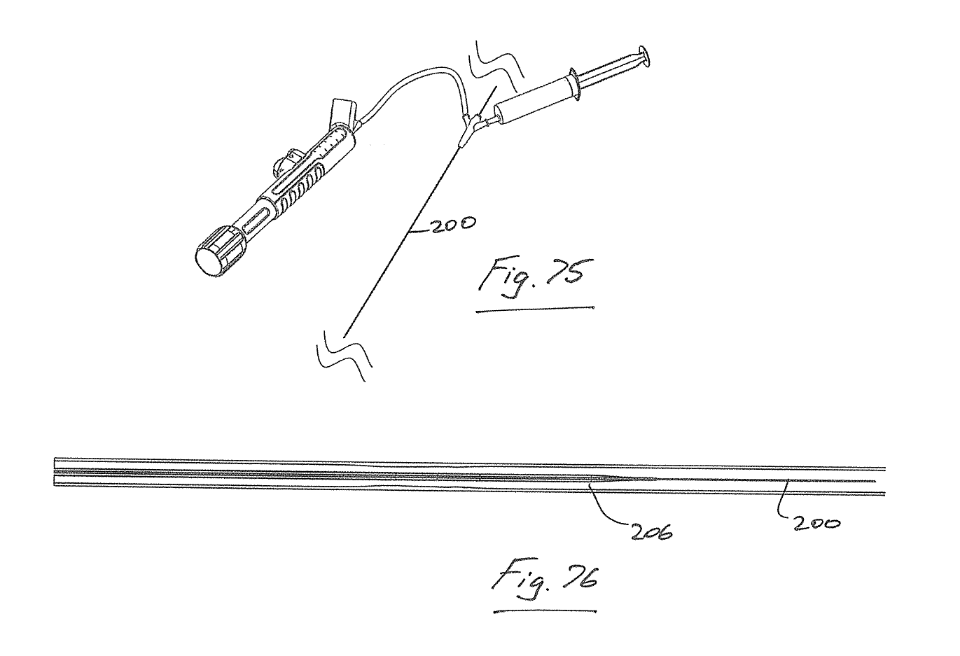

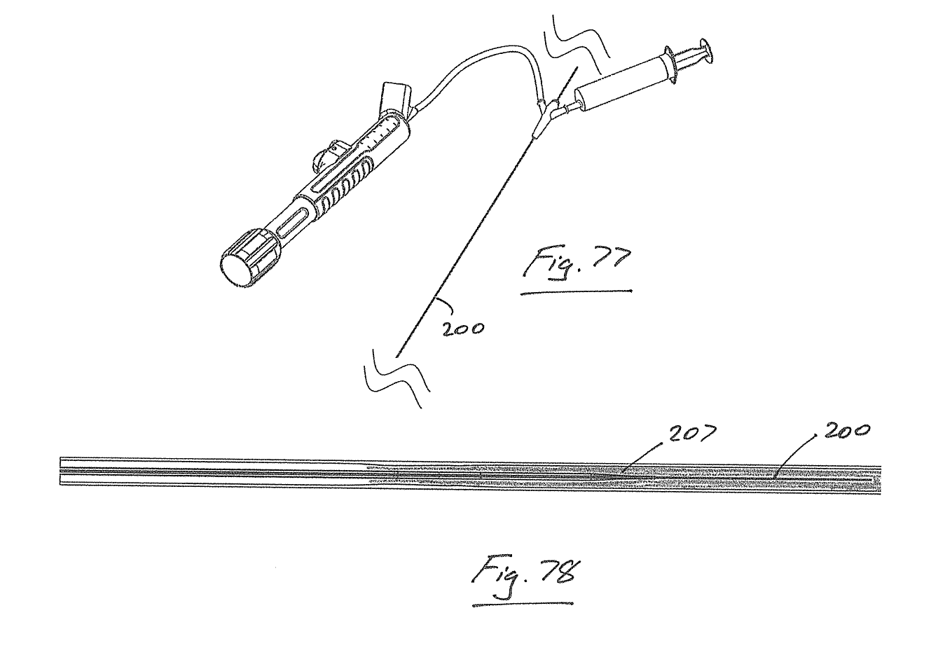

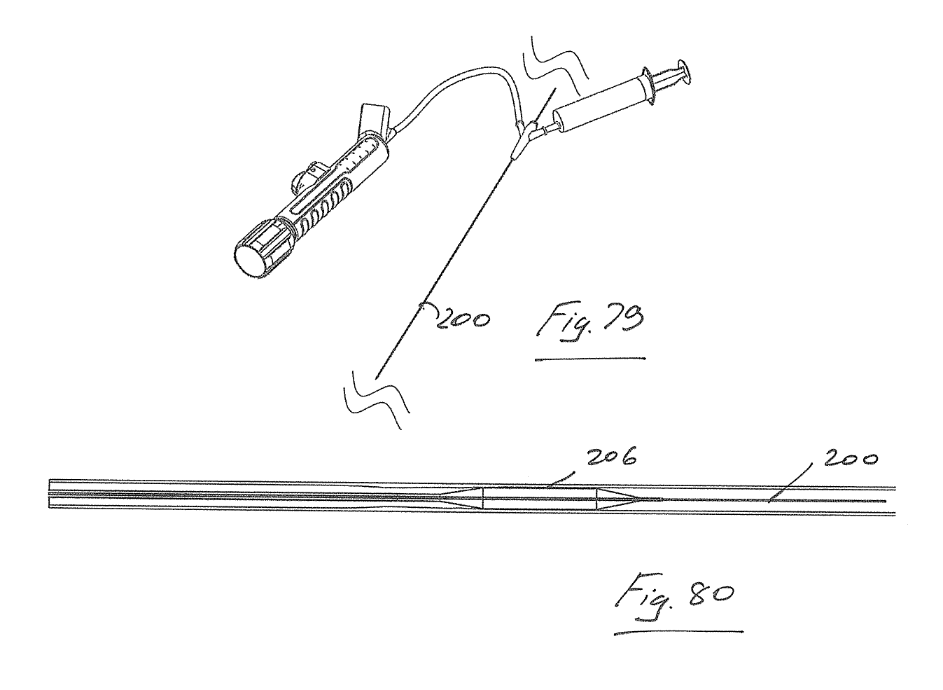

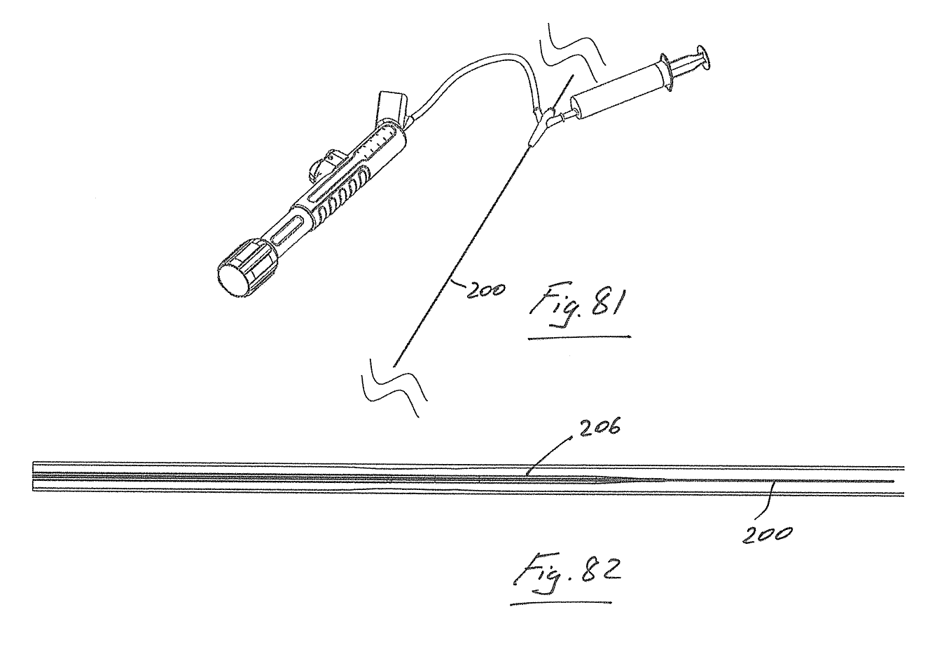

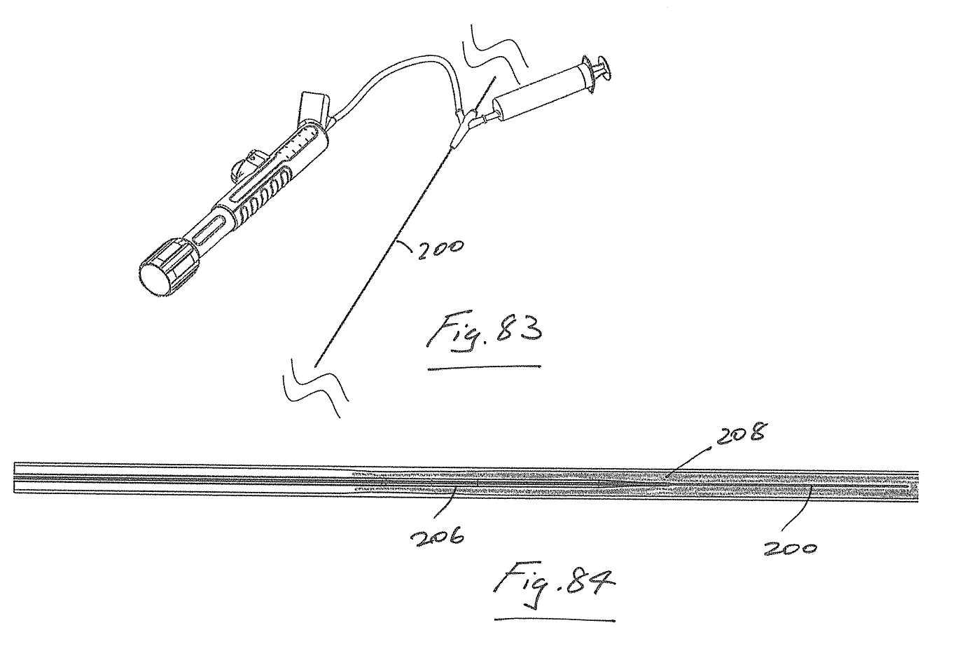

Reference is made to FIG. 67 to FIG. 84.
1. Insert a guide wire 200 across a stenosis 201 in a vessel 202 (FIG. 69,70).
2. Advance a catheter 205 over guide wire 200 (FIG. 71,72).
3. Inflate a balloon 206 (FIG. 73,74).
4. Deflate the balloon 206 (FIG. 75,76).
5. Deliver contrast media 207 through expandable lumen of catheter (FIG. 77,78).
6. Re inflate balloon 206 (FIG. 79,80).
7. Deflate balloon 206 (FIG. 81,82).
8. Deliver contrast media 208 through expandable lumen of catheter (FIG. 83,84).
9. Repeat steps 6 to 8 as necessary

### Valvuloplasty

Balloon valvuloplasty is the repair of a stenotic heart valve using an appropriately sized balloon to expand the valve. The balloon is placed into the valve that has become stiff from calcium build-up. The balloon is then inflated in an effort to increase the opening size of the valve and improve blood flow.

Orientation and position of the valvuloplasty balloon with respect to the valve is determined by means of an angiogram. To deliver visualisation contrast for the angiogram an angiographic catheter must be positioned in the ascending aorta. This requires a separate femoral artery incision and access site for the angiographic catheter, which introduces additional risk for hematoma, bleeding, and infection. Additional procedural time and cost is also incurred.

### How the Current Invention can Solve the Problem

Providing an expandable lumen on the valvuloplasty catheter would allow contrast to be delivered through the valvuloplasty balloon catheter and would negate the need for a secondary incision.

### TAVI

Transcatheter Aortic Valve Implantation (TAVI) is a medical procedure to implant an artificial aortic valve inside the patient's own aortic valve eliminating the requirement for invasive, open-heart surgery. The valve is delivered via a catheter inserted through either the femoral artery or the apex of the heart.

Orientation and position of the artificial valve is determined by means of an angiogram and echocardiography. To deliver contrast for the angiogram an angiographic catheter must be positioned in the ascending aorta. This requires a separate femoral artery incision and access site for the angiographic catheter which introduces additional risk for hematoma, bleeding, and infection. Additional procedural time and cost is also incurred.

### How the Current Invention can Solve the Problem

Providing an expandable lumen on the valvuloplasty catheter and valve delivery system would allow contrast to be delivered through the delivery system and would negate the need for a separate angiographic catheter and additional access site.

Guide Wire with Expandable Sheath (No Catheter Required)

For angiograms it is common procedure to insert a guide wire first then track the guide catheter/angiographic catheter over the wire so it is guided to the target location. The guide wire is then removed and contrast media injected.

Each removal and reinsertion of a device (wires, catheters etc.) increases the risk of vessel perforation, vessel dissection, dislodgement of existing debris, e.g. plaque, and vessel spasm. Additional procedural time and cost is also incurred.

### How the Current Invention can Solve the Problem

Providing an expandable lumen on the wire would facilitate the delivery of contrast with the wire only, would obsolete the requirement for a separate angiographic catheter and would reduce device exchanges, thus saving time and cost.

Guide Wire with Expandable Sheath (No Catheter Required)

For balloon occlusion, balloon thrombectomy and angioplasty the common procedure is to insert the wire first then track the catheter over the wire so it is guided to the target location.

This requires multiple device insertions and placements, each of which increases the risk of vessel perforation, vessel dissection, dislodgement of existing debris, (e.g. plaque), and vessel spasm. Additional procedural time and cost is also incurred.

### How the Current Invention can Solve the Problem

Providing an expandable lumen on the guide wire which consists of sections of variable or tailored expansion such that the distal end could expand to a larger diameter (effectively creating a balloon) and the proximal portion expanding to a smaller diameter (to act as a lumen for inflation of the distal portion).

### Hydrophyllic Primer—Lower Force, Lower Delivery Volume

Hydrophillic coatings are a common component on medical catheters. Hydrophilic coatings exhibit water-loving characteristics. Chemically, this means they participate in dynamic hydrogen bonding with surrounding water. In most cases, hydrophilic coatings are also ionic and usually negatively charged, which further facilitates aqueous interactions. Physically, these chemical interactions with water give rise to hydrogel materials that may exhibit extremely low coefficients of friction. Taken together, such chemical and physical characteristics describe a class of materials that are wettable, lubricious, and suitable for tailored biological interactions (reference, http://www.mddionline.com/article/hydrophilic-coatings-considerations-product-development)

This embodiment focuses on the application of a hydrophilic coating to the underside of the lumen on demand that has extended hydrophilic capabilities that ensure that there is continual surface tension between the contrast media and the internal coating on the lumen. This ensures that the minimal injection force is required to deliver the contrast.

Contrast with Balloon Catheter

Angioplasty is the technique of mechanically widening narrowed or obstructed arteries and veins. An empty and collapsed balloon on a guide wire, known as a balloon catheter, is passed into the narrowed locations and then inflated to open up the blood vessel for improved flow.

With angioplasty, multiple inflations at the same location, to ensure that the vessel has been completely opened, and multiple inflations at different points along the vessel, to treat multiple narrowing's in the vessel, may be required.

Currently the two options available to the operator for carrying out the angiogram are;
to remove the balloon catheter and inject the contrast die through the introducer sheath. Unnecessary procedural time is wasted in removing and reinserting the balloon, in conjunction with the associated radiation exposure for visualisation of the procedure. Additionally, as contrast dye is injected through the introducer sheath which is not positioned directly at the stenosis, excessive amounts of contrast dye are required to be delivered to ensure adequate angiographic images are obtained.
Reinserting guide wires can also lead to vessel perforation, vessel dissection, dislodgement of existing debris, (e.g. plaque), and vessel spasm
to remove the guide wire and inject the contrast dye through the balloon catheter guide wire lumen.
Unnecessary procedural time is wasted in removing and reinserting the guide wire in conjunction with the associated radiation exposure for visualisation of the procedure.
The balloon also has to be pulled back to ensure the tip of the balloon catheter is proximal to the stenosis prior to delivery of contrast die. This balloon then has to be repositioned for subsequent inflations, providing the potential of losing the “optimal” position.
Presence of contrast dye in the guide wire lumen can make the guide wire ‘sticky’ and restrict guide wire movement. An extra operation of flushing the guide wire lumen with saline may be required.
Reinserting guide wires can also lead to vessel perforation, vessel dissection, dislodgement of existing debris, (e.g. plaque), and vessel spasm.

### How the Current Invention can Solve the Problem

Providing an expandable lumen on the PTA catheter eliminates the need to remove either the balloon catheter or the guide wire, thus providing a feasible solution to the perceived problems outlined in a) and b) above.

Angiographic Catheters/Guide Catheters; are Essentially Hollow Tubes Used for the Delivery, and Sometimes Retrieval, of Various Substances and Materials into the Patient.

Examples of uses for these catheters are:
delivery of embolic materials such as coils, plugs, beads, foam, gel and sclerosant
delivery of contrast media for angiographic purposes
delivery of saline
delivery of drugs such as thrombolytics, anticoagulants, chemotherapy
providing guide wire support and direction
removal of fluid—cerebral, spinal etc.
removal of thrombus

A major drawback of these catheters is that they possess only one lumen.

Therefore, if the catheter is being delivered over a guide wire, the guide wire must be removed prior to delivering/removing the substance. This increases procedural duration, the contrast volume required to adequately visualise the anatomy, and the radiation exposure due to increased fluoroscopy time and may increase the risk to the patient of vessel perforation, vessel dissection, dislodgement of existing debris (e.g. plaque) and vessel spasm through increased guide wire movement.

Additionally, if more than one substance is required to be delivered through the catheter they cannot be delivered simultaneously e.g. embolisation beads and contrast. The operator requires contrast to assess whether sufficient volume of beads have been delivered to achieve adequate vessel embolisation. To deliver the contrast in between bead deliveries the operator must firstly change over the syringe from the bead filled syringe to the contrast filled syringe thereby increasing procedural time and may also risk dislodging the catheter from its original target location.

A separate step for flushing of the lumen with saline may also be required if the two materials are not compatible with one another.

### How the Current Invention can Solve the Problem

Providing an expandable lumen as a fluid delivery lumen on the catheter negates the need to remove the guide wire and provides separate lumens for delivering or extracting substances from the vasculature.

### For Delivery of Solid Materials Such as Micro Beads for Embolisation

Microbeads are delivered into vessels to embolise or block blood flow through that vessel. Many of the target vessels are small diameter (<5 mm) and can be very tortuous. For this reason a guide wire is positioned first and the guide catheter is tracked along the guide wire to the target site. Currently there is a requirement to remove the guide wire to provide a lumen in the guide catheter for bead delivery. Removing the guide wire increases the risk of the guide catheter dislodging from the target vessel, causing inaccurate delivery of the beads, potentially leading to suboptimal treatment or non target embolisation. Reinserting the guide wire also poses risks of vessel perforation, vessel dissection, dislodgement of existing debris (e.g. plaque) and vessel spasm through increased guide wire movement.

### How the Current Invention can Solve the Problem

Providing an expandable lumen on the catheter which can accommodate solid materials eliminates the need to remove the guide wire as it provides a separate lumen for delivering embolisation beads.

Anti Reflux Catheter for Embolisation Procedures, e.g. Embolisation of Benign Prostate Hyperplasia, Uterine Fibroid Embolisation, and Also for Use in Balloon Assisted Coiling Procedures.

During embolisation procedures, reflux of the embolisation material may occur when blood flow starts to slow down as vessel is being embolised, or, in a vein where antegrade and retrograde flow may already exist. To combat this, an anti reflux device is commonly used which may be in the form of an inflated balloon, nest of embolisation coils or an umbrella type anti reflux device. Some or all of these would be used in conjunction with the guiding catheter, to deliver the embolisation material, leading to extra cost for multiple devices, additional procedural time to deliver, deploy and retrieve, and the additional risks associated with inserting multiple devices.

### How the Current Invention can Solve the Problem

Providing an expandable lumen on the guide catheter which consists of sections of variable or tailored expansion such that the distal end could expand to a larger diameter (effectively creating a balloon for prevent of reflux) and the proximal portion expanding to a smaller diameter (to act as a lumen for inflation of the distal portion).

This would leave the guide catheter lumen free for delivery of the embolic agent whilst proving an anti reflux device on the same catheter to avoid use of multiple devices, without adversely affecting catheter profile.

### Stent Delivery Systems: Self Expanding, Balloon Expanding, Multiple Stent Systems—

A stent is a small mesh tube that is delivered into arteries or veins and then expanded to support the vessel wall to prevent reduction in the lumen diameter.

There are different ways a stent can be delivered.
Mounted on a balloon and deployed by expanding the balloon
Self expanding stents such as Nitinol stents are delivered within a catheter and are deployed by withdrawing the outer sheath thereby releasing the stent.
Some self expanding stent systems are capable of holding multiple stents which can be deployed sequentially.

With each of these approaches it is essential to visualise the position where the stent is located for accurate and safe delivery of the stent(s). The current approach is to take an angiographic ‘roadmap’ where the vessel is injected with contrast media and an angiogram carried out prior to the delivery catheter being placed in the vessel. This method is chosen due to difficulties with delivering contrast media while the stent delivery catheter is in place (these difficulties include delivering contrast through the sheath while the balloon catheter/stent delivery system is in place due to space constraints).

The roadmap approach does not provide real time angiographic visualisation and so movement of the patient during the procedure and changes to the vessel during the procedure cannot be identified without removing the delivery system and obtaining another angiogram.

This presents problems with each of the three approaches outlined above.
For balloon expandable stents it could be beneficial before, during and after the inflation to obtain an angiographic image to ensure correct position, adequate inflation pressure and whether or not repeat balloon inflations may be required
For self expandable stents it could be beneficial before, during and after the deployment to obtain an angiographic image to ensure correct position and whether or not follow up balloon inflations may be required to fully expand the stent
For multiple self expandable stent systems it could be beneficial before, during and after the deployment to obtain an angiographic image to ensure correct position, to ensure overlapping of stents hasn't occurred and whether or not follow up balloon inflations may be required to fully expand the stent(s)

### How the Current Invention can Solve the Problem

Providing an expandable lumen with the stent delivery catheter (be it balloon catheter of self expanding stent delivery system) negates the need to remove the catheter to obtain an angiogram thus enabling real time angiography capability during the procedure.

### Thrombectomy/Embolectomy and Catheter Directed Thrombolysis Procedures:

Thrombectomy is the minimally invasive removal of thrombi (blood clots) which are obstructing blood flow.

Balloon thrombectomy is performed by inserting a catheter with an inflatable balloon attached to its tip into a vessel, passing the catheter tip beyond the clot, inflating the balloon, and removing the clot by withdrawing the catheter, to which the clot is adhered. The catheter is called a Fogarty catheter, named after its inventor Thomas J. Fogarty.3

Aspiration thrombectomy is a procedure where the thrombus is removed by suction rather than balloon extraction.

Catheter directed thrombolysis is the delivery of lysis agents for chemically breaking down a blood clot. This may be combined with aspiration thrombectomy to remove a clot as it breaks down.

Each of the procedures requires delivery of contrast media to visualise the clot and locate the catheter under fluoroscopy. Visualisation of the treatment area would be beneficial during the thrombectomy/thrombolysis period of the procedure to ensure correct device position and whether all of the thrombus has been removed/is broken down.

As the lumens are being utilised by the guide wire, a balloon catheter, a vacuum or lysis delivery, it is not possible to deliver contrast through the catheter without disturbing some other aspect of the procedure.

### How the Current Invention can Solve the Problem

Providing an expandable lumen on the external surface of the catheter, contrast media can be delivered when required for real time visualisation of the procedure.

### Localised Delivery of Lysis/Chemo Drugs

Fluids are delivered to the body for a number of reasons e.g. cancer treating chemotherapy drug, clot busting lysis agents, contrast media for fluoroscopy.

These fluids, while necessary to treat the target area, can be harmful to other areas within the body.

### How the Current Invention can Solve the Problem

Providing an expandable lumen on the external surface of the catheter which terminates a defined distance proximal to the catheter tip provides a delivery channel for the fluid while the interior lumen ending at the distal tip of the catheter can act as an aspiration channel to remove the fluid once its function has been performed.

### Catheter for Mixing Two Materials

In some cases it can be beneficial to provide a means of mixing two materials at the target site e.g. two-part glue which sets quickly when mixed so cannot be mixed prior to delivery.

### How the Current Invention can Solve the Problem

Providing an expandable lumen on the external surface of the catheter provides a delivery channel for one of the materials while the interior lumen ending at the distal tip of the catheter acts as second channel ensuring that the two materials do not mix until they reach the distal end of the catheter.

Alternatively, the outer sheath could be composed of two expandable lumens which each carry a different fluid for mixing at the distal tip.

### Cardioplegic Solution Delivery

Cardioplegia is paralysis of the heart. This stops the heart so that surgical procedures can be performed in a still and bloodless field for procedures such as coronary artery bypass grafting and heart valve replacement amongst others.

Cardioplegia catheters incorporate between three and four lumens, one for the cardioplegia administration, one for venting the aortic root after the administration of cardioplegia, a thin pressure monitor lumen and one for balloon inflation (if balloon is present).

Due to the number of lumens in the catheter the profile is raised and steerability/trackability of the catheter can be diminished.

### How the Current Invention can Solve the Problem

Providing an expandable lumen on the external surface of the catheter provides a delivery channel for either balloon inflation or cardioplegic solution delivery whilst improving the steerability and lowering the profile of the catheter.

### 1. Pacing Lead Delivery System

Pacing leads are delivered over a wire/stylet into the chambers of the heart. This is assisted by using an image guidance roadmap, obtained at the start of the procedure. If the operator experiences difficulties in positioning the lead a real time image would assist in relieving those difficulties.

### How the Current Invention can Solve the Problem

Providing an expandable lumen on the external surface of the lead provides a delivery channel for contrast which would enable real time visualisation of the anatomy to assist in lead delivery and positioning.

### 2. Ablation Catheters

Ablation catheters deliver localised heating or cooling to destroy abnormal tissues within the body e.g. Cardiac ablation for fibrillation, tumour ablation etc.

These procedures often require visualisation of the area being treated thus requiring a separate catheter for delivery of the contrast media. Delivering the contrast media requires either a separate catheter which requires either;
a) a separate access site for the angiographic catheter which introduces risk for hematoma, bleeding, and infection. Additional procedural time and cost is also incurred.
b) Removal of the ablation catheter to allow access for the angiographic catheter, and vice versa. Reinserting catheters can lead to perforation, dissection, dislodgement of debris and vessel spasm. Additional procedural time and cost is also incurred.

### How the Current Invention can Solve the Problem

Providing an expandable lumen on the external surface of the ablation catheter provides a delivery channel for contrast which would enable real time visualisation of the anatomy and reduce the number of device exchanges within the procedure.

The invention could also be utilised for delivery of local anaesthetic or coolant during the procedure.

### 3. Lateral Fluid Delivery to Target Aneurysms

Aneurysms are localized, blood-filled balloon-like bulges in the wall of a blood vessel. They can occur in any blood vessel but most commonly in arteries at the base of the brain (the circle of Willis). An aortic aneurysm occurs in the main artery carrying blood from the left ventricle of the heart. When the size of an aneurysm increases, there is a significant risk of rupture, resulting in severe hemorrhage, other complications or death.

Where aneurysms are located in the side wall of the vessel a guide catheter must be placed into the neck of the aneurysm to deliver targeted contrast media for visualisation of the aneurysm. This can lead to risks of perforating the aneurysm wall with the guide catheter.

Another method of visualisation is to fill the vessel with contrast allowing contrast to flow into the aneurysm. This requires higher volume of contrast media and does not ensure the adequate circulation of contrast within the aneurysm.

### How the Current Invention can Solve the Problem

Providing an expandable lumen on the external surface of the guide catheter which incorporates side holes either along the length or at the tip facilitates lateral delivery of contrast which flow directly into the aneurysm without having to place the guide catheter in the aneurysm.

As the expandable lumen is collapsed and therefore low profile during delivery and retrieval, it does not significantly increase the profile of the catheter which is critical especially in small neuro vasculature.

### 4. Vacuum

There is often a requirement to remove materials from the body during interventional procedures e.g. fluid from the brain or spine, debris following plaque excision, implanting stents/valves or ballooning procedures such as balloon assisted valvuloplasty.

The common approaches to accommodating these requirements are;
a) the addition of an extra lumen for the vacuum thereby increasing the profile of the device and reducing its flexibility.
b) inserting a separate device such as a guide catheter to act as a vacuum lumen. This requires a separate access site which introduces extra risk for hematoma, bleeding, and infection

### How the Current Invention can Solve the Problem

Providing an expandable lumen on the surface of the device which expands when vacuum is applied would ensure a low profile on insertion and removal and the presence of a vacuum lumen when required.

### 5. Foley Catheter

A Foley catheter is a flexible tube that is often passed through the urethra and into the bladder. A balloon is situated near the tip of the catheter to prevent the catheter from slipping out of position.

Some Foley catheters have three lumens—irrigation, drainage and balloon inflation Some have two lumens—drainage and balloon inflation

There is a requirement to maintain the drainage lumen as large as possible to prevent clogging and to minimise the profile of the catheter for ease of entry and patient comfort

### How the Current Invention can Solve the Problem

Providing an expandable lumen on the external surface could replace either the balloon inflation lumen or the irrigation lumen thus ensuring that the catheter is lower in profile during delivery and retrieval. The expandable lumen could also be used as the drainage lumen, expanding once the pressure in the bladder is sufficient to expand the lumen.

Modification and additions can be made to the embodiments of the invention described herein without departing from the scope of the invention. For example, while the embodiments described herein refer to particular features, the invention includes embodiments having different combinations of features. The invention also includes embodiments that do not include all of the specific features described.

The invention is not limited to the embodiments hereinbefore described, which may be varied in construction and detail.

1-34. (canceled)

35. A catheter comprising a shaft extending from a proximal end to a distal end and a sheath which extends substantially along the full length of the catheter shaft, the sheath having a proximal end and a distal end and being expansile for fluid delivery from the proximal end of the sheath to the distal end of the sheath, the sheath having a contracted configuration in which the sheath lies substantially against the outer wall of the catheter shaft and an expanded delivery configuration in which at least a portion of the sheath extends radially outwardly of the catheter shaft, wherein the sheath comprises a compliant material to urge the sheath to return automatically from the expanded configuration to the contracted configuration.

36. A catheter as claimed in claim 35 wherein the sheath comprises a plurality of layers.

37. A catheter as claimed in claim 36 wherein the sheath comprises an outer layer of compliant material and an inner layer of a reinforcing material.

38. A catheter as claimed in claim 37 wherein the reinforcing material is at least partially porous.

39. A catheter as claimed in claim 37 comprising an inner sealing layer between the reinforcing layer and the outer wall of the catheter.

40. A catheter as claimed in claim 37 wherein the reinforcing layer lies between an inner sealing layer and the outer layer of compliant material.

41. A catheter as claimed in claim 36 wherein the reinforcing layer of the sheath comprises a non-compliant material.

42. A catheter as claimed in claim 35 wherein the distal end of the sheath is secured to the catheter shaft.

43. A catheter as claimed in claim 42 wherein the distal end of the sheath is bonded to the catheter shaft.

44. A catheter as claimed in claim 35 wherein the proximal end of the sheath is connected to a Luer fitting to provide access for fluid flow into the sheath.

45. A catheter as claimed in claim 44 wherein the sheath is spaced-apart from the shaft at the proximal end to define an in-flow region for fluid.

46. A catheter as claimed in claim 44 including a protector between the Luer fitting and the proximal end of the sheath, the protector may comprise a protective sleeve or a support tube.

47. A catheter as claimed in claim 45 wherein the protector extends distally of the Luer fitting at the proximal end and is adapted to provide strain relief at the connection between the Luer fitting and the sheath.

48. A catheter as claimed in claim 35 wherein the catheter shaft and the sheath are concentric in the expanded delivery configuration.

49. A catheter as claimed in claim 35 wherein the catheter shaft and the sheath are concentric in the contracted configuration and eccentric in the expanded configuration.

50. A catheter as claimed in claim 35 wherein the sheath is eccentric to the shaft in both the contracted and the expanded configuration.

51. A catheter as claimed in claim 35 wherein the catheter is generally cylindrical having an outer shaft diameter and the sheath is generally cylindrical having an inner sheath diameter and wherein the outer shaft diameter is substantially equal to the inner sheath diameter.

52. A catheter as claimed in claim 35 wherein the sheath comprises at least one fluid outlet or outlets adjacent to the distal end of the sheath, the sheath may comprise a plurality of outlets or the sheath may comprise a single outlet on one side of the sheath.

53. A catheter as claimed in claim 35 comprising a deflector element for directing fluid flow from the outlet(s), the deflector element may be adapted to deflect fluid to flow in a proximal direction.

54. A catheter as claimed in claim 35 comprising a coating on the outer surface of the catheter shaft and/or the inner surface of the sheath, the coating may be of a lubricious material.

55. A catheter as claimed in claim 35 comprising formations on the outer surface of the shaft and/or the inner surface of the sheath.
